(19) United States
(12) Patent Application Publication
(10) Pub. No.: US 2024/0269026 A1
(43) Pub. Date: Aug. 15, 2024

(54) Decubitus Prevention Device

(71) Applicant: Universal Tech Corporation, Ettrick, WI (US)

(72) Inventors: Matthew Wayne Kriesel, Melrose, WI (US); Troy Bradley Goodenough, Mindoro, WI (US)

(21) Appl. No.: 18/105,143

(22) Filed: Feb. 2, 2023

Related U.S. Application Data
(60) Provisional application No. 63/306,490, filed on Feb. 3, 2022.

(51) Int. Cl. A61G 7/057; A61B 5/00; A61H 7/00; A61H 15/00; A61H 15/02

(52) U.S. Cl. CPC A61G 7/05707; A61B 5/447; A61H 7/004; A61H 15/0078; A61H 15/02; A61H 2015/005; A61H 2201/0207; A61H 2201/0214; A61H 2201/0242; A61H 2201/1207; A61H 2201/1418; A61H 2201/1454; A61H 2205/081; A61H 2205/086

(57) ABSTRACT

An inventive decubitus prevention device comprises a thermal support member disposed upon a massage member. The massage member comprises at least one manipulation element disposed within a housing component and is configured such that the manipulation element is in contact with the bottom side of the thermal support member. The thermal support member comprises at least one thermal element at least partially encased within a polymeric member. In some embodiments, the inventive decubitus prevention device can further comprise a therapeutic member disposed upon the thermal support member. The therapeutic member comprises a cushioning polymer disposed at least upon the top side of a mitigation member. The inventive decubitus prevention device can provide a therapeutic force to a user, and can additionally provide a heating effect and/or a cooling effect to a user. The inventive decubitus prevention device can be utilized with a bed (such as a mattress combination), a wheelchair, a lounge chair, a couch, an office chair, an examination table, and the like.

### CROSS-REFERENCE TO RELATED APPLICATION

This application claims the priority benefit of U.S. Provisional Application No. 63/306,490 entitled “Decubitus Prevention Device”, which was filed Feb. 3, 2022, and which is hereby incorporated herein by reference in its entirety.

### TECHNICAL FIELD

The present disclosure relates to the prevention of decubitus ulcers. In some more particular embodiments, the present invention relates to an inventive decubitus prevention device, such as in the form of a bed mattress combination.

### BACKGROUND

Persons restricted to a particular position, such as a prone position (i.e., lying on one's back, front, side, etc.) or a sitting position, for extended periods of time (e.g., hospital patients, elderly, paralyzed persons, coma patients, burn victims, etc.) are susceptible to decubitus ulcers, which can form when pressure against the skin restricts or blocks the blood flow supplying capillaries below the skin tissue. Accordingly, such restriction or blockage of the blood flow can prevent oxygen and nutrients from reaching those areas of the skin. As a result, decubitus ulcers (e.g., bedsores, etc.) tend to form. In some situations, the presence or accumulation of heat and/or moisture at such areas of the skin can worsen the problem, including the potential development of infection. In other situations, additional heat may actually be required for the recovery and/or well-being of a particular person. In still other situation (e.g., burn victims, patients with a fever, etc.), a cooling effect may be required. In yet other situations, therapeutic forces (e.g., massage therapy) may be required. Thus, there is a need for a decubitus prevention device which can reduce or eliminate the occurrence of decubitus ulcers. There is also a need for a decubitus prevention device which can provide massage therapy or other such therapeutic actions at least to such areas of the skin that are susceptible to decubitus ulcers, such as to increase blood flow thereto. There is a further need for a decubitus prevention device which can provide or remove heat and/or remove moisture at least from such areas of the skin that are susceptible to decubitus ulcers.

### SUMMARY

In response, the invention of the present disclosure solves one or more of the problems and/or needs discussed above. More particularly, an inventive decubitus prevention device is presented. Such device may useful in the form of bed/mattress combinations, wheelchairs, lounge chairs, couches, office chairs, examination tables, and the like.

In one embodiment, the inventive decubitus prevention device can be in the form of a mattress combination comprising a thermal support member disposed upon a massage member. The massage member can comprise a housing component which can include a frame element and one or more cross member elements connected to the frame element to provide enhanced structural stability, among other things. Optionally, the housing component of the massage member can further include one or more elevation elements, which can elevate the height of the decubitus prevention device. Disposed within the housing component can be one or more manipulation elements which can apply a massaging action (e.g., a therapeutic force) upon at least a portion of a user's body in contact with the inventive decubitus prevention device. The thermal support member can comprise a thermal element at least partially embedded into a polymeric member. In some embodiments, the length and width dimensions of the polymeric member (which generally defines the dimensions of the thermal support member) will be substantially equivalent to the length and width dimensions of the massage member.

In another embodiment, the inventive decubitus prevention device can be in the form of a mattress combination comprising a thermal support member disposed upon a massage member as described above. In this embodiment, the inventive decubitus prevention device further comprises an optional therapeutic member disposed upon the thermal support member. The therapeutic member can comprise a mitigation member which can optionally be perforated to form channels at least partially through the thickness thereof. The therapeutic member further comprises a thermal-conductive polymer disposed upon the top side thereof and at least partially into the interior thereof, including into any channels which may optionally be present. In some embodiments, the therapeutic member can further comprise an optional barrier layer disposed upon the bottom side thereof and/or an optional comfort layer disposed upon the top side thereof.

In some embodiments, one or more of the components of the inventive decubitus prevention device (i.e., the massage member, the thermal support member and the optional therapeutic member) may be separately or collectively at least partially encased in a sheath component.

The invention also provides for methods of making an inventive decubitus prevention device, as well as methods for making the massage member, the thermal support member and the optional therapeutic member.

More particularly, in a first embodiment, a decubitus prevention device comprises a massage member and a thermal support member, each comprising a top side and a bottom side. The massage member comprises a housing component and at least one manipulation element, wherein the at least one manipulation element is disposed within the housing component. The thermal support member comprises a polymeric member and at least one thermal element, wherein the at least one thermal element is at least partially disposed within the polymeric member. In this first embodiment, the thermal support member is disposed upon the top side of the massage member such that the bottom side of the thermal support member is in contact with the at least one manipulation element.

In some aspects of this first embodiment, the housing component comprises a frame element and at least one cross member element. In other aspects, the massage member further comprises at least one elevation element disposed upon the bottom side thereof. In yet other aspects, the at least one manipulation element comprises a first type of massage member and a second type of massage member.

In some aspects of this first embodiment, the polymeric member comprises a viscoelastomeric and cohesive cushioning polymer. In other aspects, the cushioning polymer is formed from a reaction media comprising:
a. about 3 wt % to about 20 wt % isocyanate prepolymer or silicone prepolymer;
b. about 20 wt % to about 40 wt % polyols; and
C. about 40 wt % to about 80 wt % epoxidized triglyceride plasticizer.
   In some further aspects, the polyols comprise hydroxyl terminated polyols. In other further aspects, the polyols comprise a polybutadiene polyol. In yet other further aspects, the polyols further comprise a polyether diol. In still other further aspects, the epoxidized triglyceride plasticizer is an epoxidized soybean oil plasticizer. In yet other further aspects, the reaction media further comprises about 0.001 wt % to about 5 w % catalyst.

In some aspects of this first embodiment, the thermal support member has a 00 Shore Hardness of about 0 to about 30. In other aspects, the thermal support member further comprises a thermal element support component disposed at least partially within the polymeric member such that the thermal element support component is in contact with the at least one thermal element. In yet other aspects, the thermal support member further comprises a bottom side barrier layer disposed upon the bottom side thereof. In still other aspects, the thermal support member further comprises a top side barrier layer disposed upon the top side thereof.

In some aspects of this first embodiment, the decubitus prevention device further comprises a thermal device component connected to the at least one thermal element. In some further aspects, the thermal device component comprises a wireless user interface.

In some aspects of this first embodiment, the thermal support member is at least partially encased in a sheath member. In other aspects, the massage member and the thermal support member are collectively at least partially encased in a single sheath member.

In a second embodiment, a decubitus prevention device comprises a massage member, a thermal support member and a therapeutic member, each comprising a top side and a bottom side. The massage member comprises a housing component and at least one manipulation element, wherein the at least one manipulation element is disposed within the housing component. The thermal support member comprises a polymeric member and at least one thermal element, wherein the at least one thermal element is at least partially disposed within the polymeric member. The therapeutic member comprises a mitigation member having a top side and a bottom side and a thermal-conductive polymer. In this second embodiment, the thermal support member is disposed upon the top side of the massage member such that the bottom side of the thermal support member is in contact with the at least one manipulation element, and the therapeutic member is disposed upon the top side of the thermal support member.

In some aspects of this second embodiment, the housing component comprises a frame element and at least one cross member element. In other aspects, the massage member further comprises at least one elevation element disposed upon the bottom side thereof. In yet other aspects, the at least one manipulation element comprises a first type of massage member and a second type of massage member.

In some aspects of this second embodiment, the polymeric member comprises a viscoelastomeric and cohesive cushioning polymer. In other aspects, the cushioning polymer is formed from a reaction media comprising:
a. about 3 wt % to about 20 wt % isocyanate prepolymer or silicone prepolymer;
b. about 20 wt % to about 40 wt % polyols; and
C. about 40 wt % to about 80 wt % epoxidized triglyceride plasticizer.
   In some further aspects, the polyols comprise hydroxyl terminated polyols. In other further aspects, the polyols comprise a polybutadiene polyol. In yet other further aspects, the polyols further comprise a polyether diol. In still other further aspects, the epoxidized triglyceride plasticizer is an epoxidized soybean oil plasticizer. In yet other further aspects, the reaction media further comprises about 0.001 wt % to about 5 w % catalyst.

In some aspects of this second embodiment, the thermal support member has a 00 Shore Hardness of about 0 to about 30. In other aspects, the thermal support member further comprises a thermal element support component disposed at least partially within the polymeric member such that the thermal element support component is in contact with the at least one thermal element. In yet other aspects, the thermal support member further comprises a bottom side barrier layer disposed upon the bottom side thereof. In still other aspects, the thermal support member further comprises a top side barrier layer disposed upon the top side thereof.

In some aspects of this second embodiment, the decubitus prevention device further comprises a thermal device component connected to the at least one thermal element. In some further aspects, the thermal device component comprises a wireless user interface.

In some aspects of this second embodiment, the mitigation member comprises at least one aperture. In some further aspects, the at least one aperture is disposed upon the top side of the mitigation member and extends at least partially through the mitigation member to form at least one channel. In other further aspects, the at least one channel comprises the thermal-conductive polymer at least partially disposed therein. In yet other further aspects, the therapeutic member comprises the thermal-conductive polymer disposed upon the top side of the mitigation member. In still other further aspects, therapeutic member further comprises the thermal-conductive polymer disposed at least partially through the mitigation member. In yet other further aspects, the therapeutic member further comprises a layer of the thermal-conductive polymer disposed upon the top side of the mitigation member.

In some aspects of this second embodiment, the thermal-conductive polymer is formed from a reaction media comprising:
a. about 2 wt % to about 20 wt % prepolymer;
b. about 1 wt % to about 65 wt % straight chain polyols;
c. about 3 wt % to about 50 wt % crosslinking polyols;
d. about 40 wt % to about 80 wt % epoxidized triglyceride plasticizer; and
e. 0 wt % to about 40 wt % viscosity reducing plasticizer.
   In some further aspects, the prepolymer is selected from the group consisting of isocyanate prepolymer and silicone prepolymer. In other further aspects, the straight chain polyols comprise polyether diols and the crosslinking polyols comprise polyether triols. In yet other further aspects, the epoxidized triglyceride plasticizer comprises epoxidized soybean oil plasticizer. In still other further aspects, the viscosity reducing plasticizer comprises ester plasticizer. In other aspects, the reaction media further comprises about 0.001 wt % to about 5 w % catalyst.

In some aspects of this second embodiment, the therapeutic member further comprises a barrier layer disposed upon the bottom side thereof. In other aspects, the therapeutic member further comprises a comfort layer disposed upon the top side thereof.

In some aspects of this second embodiment, at least one of the thermal support member and the therapeutic member is at least partially encased in a sheath member. In other aspects, the massage member, the thermal support member and the therapeutic member are collectively at least partially encased in a single sheath member.

In addition, a method of preparing a decubitus prevention device comprises:
a. providing a massage member having a top side and a bottom side comprising a housing component and at least one manipulation element, wherein the at least one manipulation element is disposed within the housing component;
b. providing a thermal support member having a top side and a bottom side comprising a polymeric member and at least one thermal element, wherein the at least one thermal element is at least partially disposed within the polymeric member; and
c. disposing the thermal support member upon the top side of the massage member such that the bottom side of the thermal support member is at least in partial contact with the at least one thermal element to form the decubitus prevention device.

In other aspects, method further comprises:
d. providing a therapeutic member having a top side and a bottom side comprising a mitigation member and a thermal-conductive polymer, wherein the thermal-conductive polymer is at least partially disposed within the mitigation member; and
e. disposing the therapeutic member upon the top side of the thermal support member.

Numerous other features and advantages of the present invention will appear from the following description. In the description, reference is made to exemplary embodiments of the invention. Such embodiments do not represent the full scope of the invention. Reference should therefore be made to the claims herein for interpreting the full scope of the invention. In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

### FIGURES

The foregoing and other features, aspects and advantages of the present invention will become better understood with regard to the following description, appended claims and accompanying drawings where:

FIG. 1A is a perspective view showing a non-limiting exemplary embodiment of an inventive decubitus prevention device of the present disclosure in the form of a mattress combination comprising a thermal support member and a massage member;

FIG. 1B is a cross-sectional side view of the inventive decubitus prevention device of FIG. 1A taken along line A-A;

FIG. 2A is a perspective view showing a non-limiting exemplary embodiment of a thermal support member component of an inventive decubitus prevention device;

FIG. 2B is a cross-sectional side view showing the thermal support member of FIG. 2A taken along line B-B;

FIG. 3A is a perspective view showing a non-limiting exemplary embodiment of a massage member component of an inventive decubitus prevention device;

FIG. 3B is a cross-sectional side view of the massage member taken along line C-C;

FIG. 4A is a perspective view showing a non-limiting exemplary embodiment of an inventive decubitus prevention device of the present disclosure in the form of a mattress combination comprising a mitigation member, a thermal support member and a massage member;

FIG. 4B is a cross-sectional side view of the inventive decubitus prevention device of FIG. 5A taken along line D-D;

FIG. 5A is a perspective view showing a non-limiting exemplary embodiment of a mitigation member;

FIG. 5B is a cross-sectional side view showing the mitigation member of FIG. 6A taken along line E-E;

FIG. 6A is a perspective view showing a non-limiting exemplary embodiment of a therapeutic member; and

FIG. 6B is a cross-sectional side view showing the therapeutic member of FIG. 7A taken along line F-F.

Repeated use of reference characters in the present specification and drawings is intended to represent the same or analogous features or elements of the present invention. It should be understood that the drawings herein are not intended to be drawn to scale, but rather are drawn to show particular elements of the invention.

### Test Methods

### 00 Shore Hardness Test Utilizing a Durometer

The 00 Shore Hardness can be determined using a durometer in accordance with ASTM D2240-00.

### Definitions

It should be noted that, when employed in the present disclosure, the terms “a” and “an” are intended to mean “at least one” of any stated features, elements, integers, steps, components, or groups and are not intended to be limited to only one of such features, elements, integers, steps, components, or groups thereof, except where specifically stated as such. In addition, use of the phrase “at least one” is not intended to render other uses of the terms “a” or “an” to be limited to only one of a feature, element, integer, step, component, or group.

It should be noted that, when employed in the present disclosure, the terms “comprises,” “comprising” and other derivatives from the root term “comprise” are intended to be open ended terms that specify the presence of any stated features, elements, integers, steps, components, or groups, and are not intended to preclude the presence or addition of one or more other features, elements, integers, steps, components, or groups thereof.

As used herein, the term “cell” refers to a cavity contained in foam.

As used herein, the term “cell connectivity” refers to a circumstance wherein at least one wall of a cell membrane of a foam surrounding the cell has orifices or pores that connect to an adjacent cell, such that an exchange of fluid is possible between such adjacent cells.

As used herein, the term “closed cell” refers to a cell in a foam wherein the cell membrane surrounding the cavity is not broken and has all membranes intact.

As used herein, the term “catalytic amount” is a term of art which is recognized by persons having ordinary skill in the art and refers to an amount that is enough to obtain a desired response or result.

As used herein, the terms “cohesive” and “cohesiveness” refer to the ability of a polymer to return to its original innate form upon subjection and subsequent removal of a stretching or compression force.

As used herein, the term “effective amount” refers to the amount required to obtain a desired result.

As used herein, the terms “elastomer,” “elastomeric,” and “elastic” are used interchangeably and refer to material having elastomeric or rubbery properties. Elastomeric materials, such as thermoplastic elastomers and thermoplastic vulcanizates for example, are generally capable of recovering their shape after deformation when the deforming force is removed. More particularly, as used herein, elastomeric is meant to be that property of any material which upon application of an elongating force in the x-y planar dimensions permits that material to be stretchable to a stretched length which is greater than its relaxed length, and that will cause the material to substantially recover its elongation upon release of the stretching elongating force. In addition to a material being elastomeric in the described x-y planar dimensions of a structure, including a substrate, the material can be elastomeric in the z planar dimension. More particularly, when a compression force is applied to a structure, that structure displays elastomeric properties and will essentially recover to its original form upon relaxation.

As used herein, the term “expand” includes not only expansion by volume, but also extension through elastomeric and stretchable planar dimension properties.

As used herein, the term “foam formulation” refers to the base resin and any additives that are combined and used in the foam-making process. The term “foam melt” refers to the mixture of components of the foam formulation after the mixture has been heated, but prior to cooling and setting of the mixture. The term “foam” and “foam composite” are used interchangeably to refer to the cooled and set mixture from the foam-making process. As used herein, the composition of the foam is considered to be generally equivalent to the composition of the foam formulation.

As used herein, the term “meltblown” refers to nonwoven materials and substrates formed by extruding a molten thermoplastic material through a plurality of fine, usually circular, die capillaries as molten threads or filaments into converging high velocity gas (e.g., air) streams which attenuate the filaments of molten thermoplastic material to reduce their diameter, which may be to microfiber diameter. Thereafter, the filaments are carried by the high velocity gas stream and are deposited on a collecting surface to form a web of randomly dispersed filaments.

As used herein, the terms “nonwoven” and “nonwoven web” refer to materials and substrates having a structure of individual fibers or filaments which are interlaid, but not in an identifiable manner as in a knitted fabric. The terms “fiber” and “filament” are used herein interchangeably. Nonwoven materials and substrates can be formed by many processes (e.g., meltblowing processes, spunbonding processes, air laying processes, bonded-carded web processes, etc.).

As used herein, the term “open-cell” refers to any cell in a foam that has at least one broken or missing membrane or an orifice in a membrane such that it is in communication with a neighboring cell.

As used herein, the term “polymer” generally includes but is not limited to, homopolymers, copolymers, including block, graft, random and alternating copolymers, terpolymers, etc., and blends and modifications thereof. Furthermore, unless otherwise specifically limited, the term “polymer” shall include all possible molecular geometrical configurations of the material. These configurations include, but are not limited to isotactic, syndiotactic and atactic symmetries. As used herein, the composition of the polymer is considered to be generally equivalent to the composition of the polymeric reaction media.

As used herein, the terms “reaction media” and “polymeric reaction media” refer to the polymer formula and any additives that are combined and used in the polymer-making process. The term “uncured” refers to a reaction media in a liquid state prior to a reaction of the constituents therein. The term “partially-cured” refers to a reaction media in a liquid or semi-liquid state wherein a reaction of the constituents has commenced, but prior to completion of the reaction.

As used herein, the term “reaction product” refers to the resulting product obtained upon curing a reaction media to form a polymer of the present disclosure.

As used herein, the term “spunbond” refers to nonwoven materials and substrates comprising small diameter fibers which are formed by extruding molten thermoplastic materials as filaments from a plurality of fine capillaries of a spinnerette having a circular or other configuration, with the diameter of the extruded fibers then being rapidly reduced. Spunbond fibers are quenched and generally not tacky when they are deposited onto a collecting surface to form a nonwoven material or substrate. Spunbond fibers are generally continuous and often have average deniers larger than about 0.3 denier, more particularly, between about 0.6 and 10 denier.

As used herein, the term “surfactant” refers to a chemical component that affects the surface tension of fluids.

As used herein, the term “thermoplastic” describes a material that softens and/or flows when exposed to heat and which substantially returns to its original hardened condition when cooled to room temperature.

As used herein, the term “thermoset” refers to a material that is capable of becoming permanently cross-linked, and the physical form of the material cannot be changed by heat without a breakdown of chemical bonds.

As used herein, the terms “viscoelastomeric” and “viscoelastic” can be used interchangeably to refer to a substance (such as a polymer) having viscous and elastic properties and which exhibits viscous flow elastic properties (as opposed to densifying compressive elastic properties, such as with foam or rubber) to return to its original innate form upon subjection and subsequent removal of a stretching or compression force.

These terms may be defined with additional language in the remaining portions of the specification.

### DETAILED DESCRIPTION

The invention is generally directed to inventive decubitus prevention devices wherein at least a portion of a user's body remains in contact with such devices for relatively prolonged periods of time, such as a bed, a wheelchair, etc. Such devices can be useful for preventing decubitus ulcers, providing pain relief, providing bodily therapy, etc. In some preferred embodiments, an inventive decubitus prevention device of the present disclosure can comprise a massage member, a thermal support member and an optional therapeutic member.

Although several exemplary embodiments of the present invention will be described herein, it should be understood that the disclosed embodiments are intended merely as non-limiting examples of the invention that may be embodied in various forms. Therefore, specific details disclosed herein, such as relating to structure, function, and the like, are not to be interpreted as limiting in any manner whatsoever, but rather only as one of numerous example bases for claims and/or teaching persons having ordinary skill in the art to variously employ the present invention in virtually any appropriately detailed structure or circumstance.

Accordingly, in the interest of brevity and conciseness, descriptions herein may be substantially directed to the non-limiting exemplary inventive decubitus prevention device in the form of a bed or mattress combination. However, it should be understood that the concepts and variations of the embodiments disclosed herein are also intended to be applied to other suitable devices, including but not limited to wheelchairs, lounge chairs, couches, office chairs, examination tables, and the like.

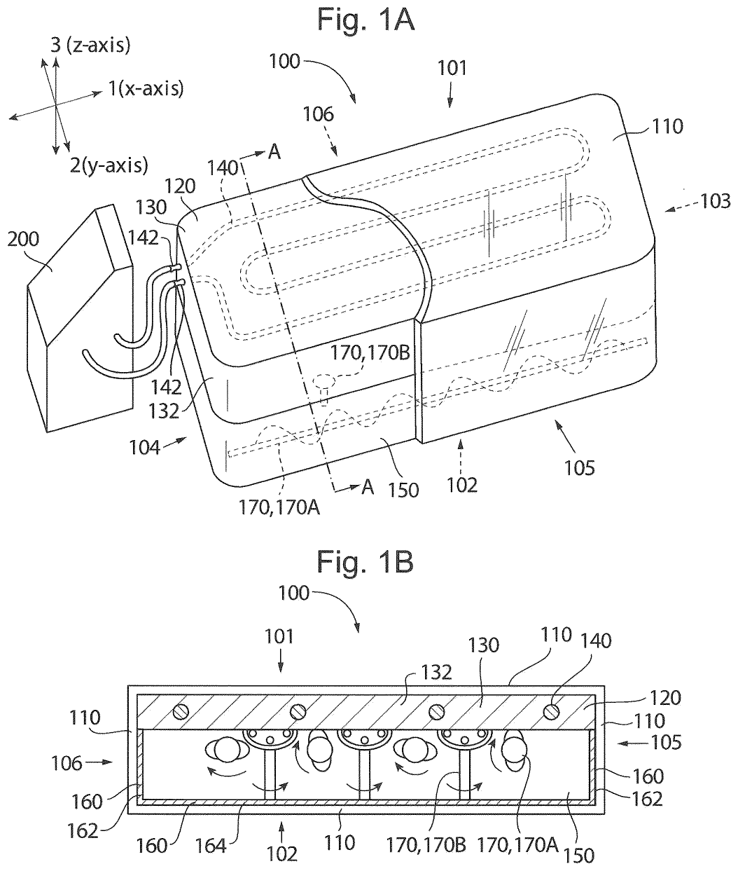

To gain a better understanding of the present invention, attention is directed to FIGS. 1A-6B for exemplary purposes showing non-limiting embodiments of an inventive decubitus prevention device 100 of the present disclosure in the representative form of a bed or mattress combination. With particular reference to FIGS. 1A-1B, one embodiment of the inventive decubitus prevention device 100 comprises a thermal support member 120 disposed upon a massage member 150. In some aspects of such embodiments, the inventive decubitus prevention device 100 can additionally comprise an optional sheath member 110 (e.g., a mattress covering, etc.) which can at least partially cover or encase the thermal support member 120 and/or the massage member 150. Such sheath member 110 can, inter alia, provide an aesthetically pleasing surface feel to the device 100, provide an aesthetically pleasing visual appearance to the device 100, help prevent potential leakage (if any) of plasticizer from the polymeric member 130 component of thermal support member 120, reduce the level of any sound emitted by the device 100, assist with maintaining the positions of the thermal support member 120 and the massage member 150 with respect to each other, and present the device 100 as a single unit, for example. Suitable materials for use as a sheath member 110 include those known to persons having ordinary skill in the art, such as plastics (e.g., polyethylene, polypropylene, PVC, etc.), polyester, elastic webbing, Damask, Spandex, satin, Sateen, vinyl, thermoplastic foam, thermoset foam, Gortex, nonwovens (e.g., meltblown, spunbond, etc.), coated or uncoated woven textiles, cotton, wool, felt, natural and synthetic leather, natural and synthetic rubbers, and the like, and combinations thereof. Preferably, such sheath member 110 will have a flexibility that is at least equivalent to, or greater than, that of the thermal support member 120 component of the inventive decubitus prevention device 100, though it need not be. The optional sheath member 110 can comprise any functional thickness, but will typically be relatively thin, such as between about 0.5 mm and about 10 mm. However, it should be understood that an optional sheath member 110 can have a thickness of less than 0.5 mm or greater than 10 mm without departing from the scope of the invention.

In some embodiments, the inventive decubitus prevention device 100 can also comprise additional layers (e.g., nonwoven layers, foam layers, felt layers, etc.) (not shown) which may be disposed atop the thermal support member 120, beneath the massage member 150, between the thermal support member 120 and the a massage member 150, and/or along the side portions of the thermal support member 120 and/or the massage member 150, without departing from the scope of the invention.

As illustrated, the inventive decubitus prevention device 100 generally comprises a generally horizontal first or top major planar side 101, an opposing generally horizontal second or bottom major planar side 102 distal to the first side 101, a vertical third or “head” (i.e., of a prone human) generally planar side or end 103 disposed orthogonally between the first side 101 and the second side 102, an opposing vertical fourth or “foot” (i.e., of a prone human) generally planar side or end 104 distal to the third side 103, a vertical fifth generally planar side 105 disposed orthogonally between the first side 101 and the second side 102 and between the third side 103 and the fourth side 104, and an opposing vertical sixth generally planar side 106 distal to the fifth side 105.

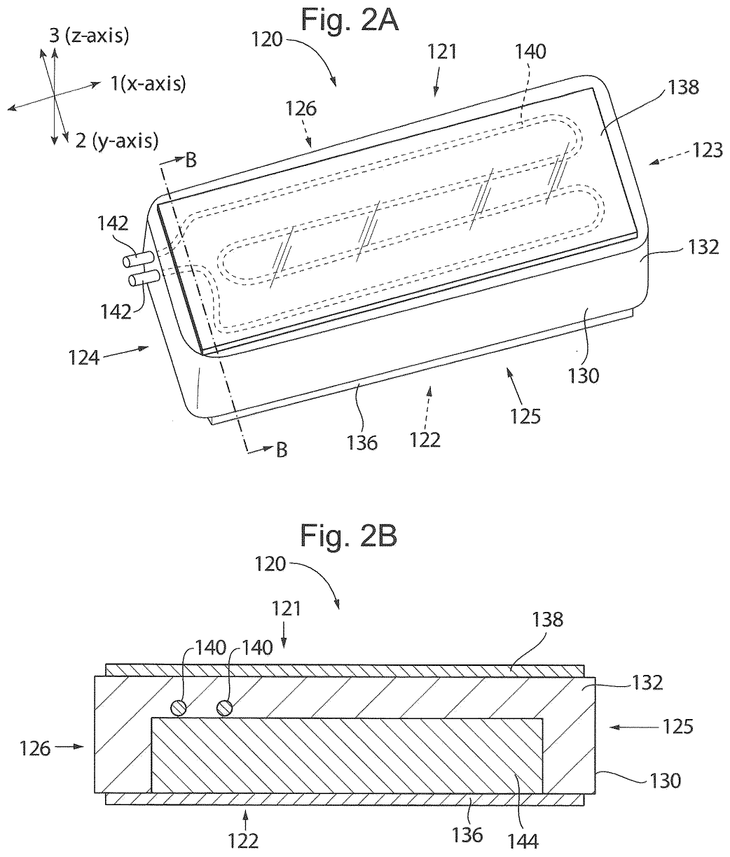

With additional reference to FIGS. 2A-2B, the thermal support member 120 component of the inventive decubitus prevention device 100 comprises a polymeric member 130 and at least one thermal element 140 disposed therein. The thermal support member 120 comprises a generally horizontal first or top major planar side 121, an opposing generally horizontal second or bottom major planar side 122 distal to the first side 121, a vertical third or “head” generally planar side or end 123 disposed orthogonally between the first side 121 and the second side 122, an opposing vertical fourth or “foot” generally planar side or end 124 distal to the third side 123, a vertical fifth generally planar side 125 disposed orthogonally between the first side 121 and the second side 122 and between the third side 123 and the fourth side 124, and an opposing vertical sixth generally planar side 126 distal to the fifth side 125. In some preferred embodiments, the thermal support member 120 can have dimensions in the x-y plane that are substantially equivalent to the dimensions in the x-y plane of the overall decubitus prevention device 100. However, it should be understood that the thermal support member 120 can have dimensions along the x-axis 1 or the y-axis 2 which are less than, or greater than, the dimensions in the x-y plane of the overall decubitus prevention device 100 without departing from the scope of the invention. In addition, the top side 121 of the thermal support member 120 may have a generally flat surface, or may comprise contours or other three-dimensional (i.e., non-flat) surface characteristics without departing from the scope of the invention.

In some preferred embodiments, the thermal support member 120 comprises a polymeric member 130 and a thermal element 140, wherein the thermal element 140 is substantially encased within the polymeric member 130 (except for extended portions 142 (i.e., exterior extending portions) of the thermal element 140).

The thickness (i.e., height as measured along the z-axis 3) of the polymeric member 130 (and thus, in some preferred embodiments, the thickness of the thermal support member 120) is not limited to any particular measurement, and will be dependent upon numerous factors including, inter alia, the size (e.g., overall outer diameter) of the thermal element 140 disposed therein, the hardness and/or flexibility of the thermal element 140 (e.g., to obtain a desired overall softness of the thermal support member 120), the softness and/or flexibility of the cushioning polymer 132 component, the degree of support desired for a user, the desired buffering effect between the massage member 150 and the user, the desired thickness and/or softness of the overall decubitus prevention device 100, etc. Desirably, the thickness of the polymeric member 130 will be sufficient to at least substantially encase the thermal element 140, though it need not be without departing from the scope of the invention. In some non-limiting exemplary embodiments, the polymeric member 130 can have a thickness such that it extends between about 1 mm to about 15 mm above the top side of the thermal element 140. However, it should be understood that the polymeric member 130 can extend less than 1 mm or greater than 15 mm above the top side of the thermal element 140 without departing from the scope of the invention. In general, the overall thickness of the polymeric member 130 will typically range between 1 cm and 20 cm. However, it should be understood that the thickness of the polymeric member 130 can be less than 1 cm or greater than 20 cm without departing from the scope of the invention. In addition, the thickness of the polymeric member 130 may or may not be uniform across the length (x-axis 1) and/or width (y-axis 2) thereof.

The polymeric member 130 component of the thermal support member 120 comprises a cushioning polymer 132, which is preferably a relatively soft, viscoelastomeric and cohesive polymer. However, any suitable polymer may be utilized for the polymeric member 130 of the thermal support member 120 provided the polymer (and resulting thermal support member 120) has a 00 Shore Hardness of about 30 or less, such as about 0 to about 30, as measured by the 00 Shore Hardness Test utilizing a durometer. In some preferred embodiments, the cushioning polymer 132 is also thermally conductive.

In some non-limiting exemplary embodiments, a suitable cushioning polymer 132 can include those polymers described in U.S. Pat. No. 7,041,719 to Kriesel et al. entitled “Shock Absorbing Compound”, the contents of which are incorporated herein by reference in a manner that is consistent herewith. Variations of such polymers, as well as other polymers having similar properties, including silicone-based polymers, can also be suitable for the present invention without departing from the scope of the invention.

By way of a non-limiting example only, a polymeric reaction media for formulating a suitable cushioning polymer 132 can be prepared comprising about 3 percent by weight of the total reaction media weight (wt %) to about 20 wt % isocyanate prepolymer, about 20 wt % to about 40 wt % polyols, and greater than 40 wt % epoxidized triglyceride plasticizer. In one more particular non-limiting example, the cushioning polymer 132 component of the polymeric member 130 could be a viscoelastomeric polymer formed from a reaction media comprising about 4 wt % to about 20 wt % prepolymer, about 20 wt % to about 40 wt % hydroxyl functional polyols, and about 40 wt % to about 80 wt % epoxidized triglyceride plasticizer. In some aspects, the prepolymer can comprise an isocyanate prepolymer or a silicone prepolymer. In some aspects, the polyols can comprise a polybutadiene polyol. In some further aspects, the polyols can further comprise a diol (e.g., polyether diol). In some aspects, the epoxidized triglyceride plasticizer can comprise an epoxidized vegetable oil plasticizer (e.g., epoxidized soybean oil plasticizer). In some aspects, the reaction media can be reacted in the presence of about 0.001 wt % to about 5 wt % catalyst (e.g., a tin based catalyst).

As referenced above, the cushioning polymer 132 of the present disclosure can comprise a prepolymer. Various prepolymers can be utilized provided they do not substantially hinder the desired cohesiveness, viscoelasticity, and shock-attenuating attributes of the polymer. In some preferred embodiments, the prepolymer can be an isocyanate. Suitable isocyanates include, inter alia, aliphatic, cycloaliphatic, aromatic and heterocyclic polyisocyanates. While not intended to be limiting, specific examples include aromatic diisocyanates (e.g., diphenylmethane diisocyanate, methylene diphenyl diisocyanate (MDI), toluene diisocyanate (TDI), etc.), aliphatic diisocyanates (e.g., hexamethylene diisocyanate (HDI), isophorone diisocyanate (IPDI), etc.) ethylene diisocyanate, 1,4-tetramethylene diisocyanate, 1,6-hexamethylene diisocyanate, 1,12-dodecane diisocyanate, cyclobutane-1,3-diisocyanate, cyclohexane-1,3- and 1,4-diisocyanate and mixtures of these isomers, 1-isocyanato-3,3,5-trimethyl-5-isocyanato methyl cyclohexane, 2,4- and 2,6-hexahydrotolylene diisocyanate and mixtures of these isomers, perhydro-2,4′- and/or 4,4′-diphenylmethane diisocyanate, 1,3- and 1,4-phenylene diisocyanate, 2,4- and 2,6-tolylene diisocyanate and mixtures of these isomers, diphenylmethane-2,4′- and/or 4,4′-diisocyanate, naphthyl-ene-1,5-diisocyanate, triphenylmethane-4,4′,4″-triisocyanate, and polyphenylpolymethylene polyisocyanates of the type obtained by condensing aniline with formaldehyde, followed by phosgenation. Example isocyanates can include prepolymers based on methylene diphenyl isocyanate reacted with polyoxyethylene/polyoxypropylene. These materials are known by such tradenames as ELASTOCAST TQZ-P23, available from BASF Corporation, having a place of business located in Florham Park, New Jersey, U.S.A., ISONATE 2181 available from Dow Chemical Company having a place of business located in Midland, Michigan, U.S.A., MONDUR MP210 available from Bayer having a place of business located in Leverkusen, Germany, and RUBINATE 1209 and RUBINATE 1790 available from Huntsman Corporation having a place of business located in Salt Lake City, Utah, U.S.A.

As referenced above, the cushioning polymer 132 of the present disclosure can also comprise a polyol component. Such polyol component can be comprised of most any polymeric compound having elastomeric properties and functional alcohol groups. For example, suitable polymeric components include, inter alia, polydienes. An example polydiene includes polybutadiene. Typically, the polybutadiene is a low molecular weight hydroxyl terminated polybutadiene resin such as POLY BD R45 HTLO, available from Cray Valley, having a place of business located in Exton, Pennsylvania, U.S.A. Such polyols have primary allylic alcohol groups that exhibit high reactivity in condensation polymerization reactions.

As referenced above, the cushioning polymer 132 of the present disclosure can also comprise an epoxidized triglyceride plasticizer, such as epoxidized animal oils and epoxidized vegetable oils. For example, suitable epoxidized vegetable oil plasticizers include, inter alia, epoxidized soybean oil, epoxidized linseed oil, epoxidized tall oil, epoxidized corn oil, epoxidized cottonseed oil, epoxidized perilla oil, epoxidized safflower oil, and the like. Epoxidized animal oils and epoxidized vegetable oils are typically obtained by the epoxidation of triglycerides of unsaturated fatty acids and are made by epoxidizing the reactive olefin groups of the naturally occurring triglyceride oils. Typically, the olefin groups are epoxidized using a peracid. One particular example of a suitable epoxidized triglyceride plasticizer is PARAPLEX G-62 available from Hallstar, having a place of business located in Chicago, Illinois, U.S.A., which is a high molecular weight epoxidized soybean oil on a carrier having an auxiliary stabilizer for a vinyl group. It has been discovered herein that PARAPLEX G-62 can function as both a plasticizer and a processing aid.

In some embodiments, the reaction media from which the cushioning polymer 132 is derived can be reacted in the presence of a catalyst or activator. Suitable catalysts include, inter alia, tertiary amines (e.g., bis(dimethylaminoethyl) ether, trimethylamine, triethylamine, N-methylmorpholine, N-ethylmorpholine, N,N-dimethylbenzylamine, N,N-dimethylethanolamine, N,N,N′,N′-tetramethyl-1,3-butanediamine, triethanolamine, 1,4-diazabicyclo[2,2,2]octane, N,N-dimethylcyclohexylamine, N-methyldicyclohexylamine, 1,8-diazabicyclo[5,4,0]-undecene-7 and its salts such as phenol salt, hexanoate, and oleate, 2,4,6-tris(diaminomethyl) phenol, and the like), tertiary phosphines (e.g., trialkylphosphines, dialkylbenzylphosphines, and the like), strong bases (e.g., alkali and alkaline earth metal hydroxides, alkoxides, and phenoxides), acidic metal salts of strong acids (e.g., ferric chloride, stannic chloride, stannous chloride, antimony trichloride, bismuth nitrate and chloride, and the like), chelates of various metals (e.g., those obtained from acetylacetone, benzoylacetone, trifluoroacetylacetone, ethyl acetoacetate, salicylaldehyde, cyclopentanone-2-carboxylate, acetylacetone-imine, bis-acetylacetonealkylenediimines, salicylaldehydeimine, and the like, with various metals such as Be, Mg, Zn, Cd, Pb, Ti, Zr, Sn, As, Bi, Cr, Mo, Mn, Fe, Co, Ni, or such ions as MoO++, UO++, and the like), alcoholates and phenolates of various metals (e.g., Ti(OR), Sn(OR), Al(OR), and the like, wherein R is alkyl or aryl and the reaction products of alcoholates with carboxylic acides, beta-diketones, and 2-(N,N-dialkylamino)alkanols, such as the well-known chelates of titanium), salts of organic acids with a variety of metals (e.g., alkali metals, alkaline earth meals, Al, Sn, Pb, Mn, Co, Ni, and Cu, including, for example, sodium acetate, potassium laurate, calcium hexanoate, stannous acetate, stannous octoate, stannous oleate, lead octoate, metallic driers such as manganese and cobalt naphthenate, and the like), and organometallic derivates of tetravalent tin, trivalent and pentavalent As, Sb, and Bi and metal carbonyls of iron and cobalt, mercury compounds (e.g., arylmercury carboxylates, phenylmercury acetate and propionate, and the like).

Typically, the catalyst is an alkyl tin compound such as dialkyltin salts of carboxylic acids, (e.g., dibutyltin diacetate, dibutyltin dilaurate, dibutyltin maleate, dilauryltin diacetate, dioctyltin diacetate, dibutyl-tin-bis(4-methylaminobenzoate), dibutyltin-bis(6-methylaminocaproate), etc.), dialkyltin mercaptides (e.g., diakyltin dimercaptide carboxylic acid esters, etc.), trialkyltin hydroxide, dialkyltin oxide, dialkyltin dialkoxide, dialkyltin dichloride, and the like. Examples of these compounds include, inter alia, trimethyltin hydroxide, tributyltin hydroxide, trioctyltin hydroxide, dibutyltin oxide, dioctyltin oxide, dilauryltin oxide, dibutyltin-bis(isopropoxide), dibutyltin-bis-(2-dimethylaminopentylate), dibutyltin dichloride, dioctylin dichloride, etc.). One particular example of an alkyl tin compound is COTIN 430, a dioctyltin carboxylate available from Cambrex Co. of Itasca, Illinois, U.S.A., which is a liquid organotin catalyst that is less sensitive to moisture and initiates relatively slower than other organotin catalysts.

In some embodiments, the cushioning polymer 132 of the present disclosure can comprise one or more additional additives. Such additional additives can include, inter alia, fillers, pigments, surfactants, additional plasticizers, organic blowing agents, stabilizers, and the like. For example, in the case where the reaction media comprises a blowing agent, the cushioning polymer 132 can be present in the form of a polymer foam. Suitable blowing agents include, inter alia, water, a chemically participating extender, carbon dioxide-producing agents, organic agents (e.g., trichlorofluoromethane, methylene chloride, low boiling hydrocarbons, ethers, ketones, etc.), and the like.

Particularly in the manufacture of foams, the reaction media can comprise surface-active additives such as emulsifiers and foam stabilizers. Suitable emulsifiers include, inter alia, sodium salts of castor oil sulfonates, salts of fatty acids with amines (e.g., oleic acid diethylamine, stearic acid diethanol amine, etc.), alkali or ammonium salts of sulfonic acids (e.g., dodecyl benzene sulfonic acid, dinaphthylmethane disulfonic acids, etc.), alkali or ammonium salts of fatty acids (e.g., ricinoleic acid, etc.), polymeric fatty acids, and the like. Suitable foam stabilizers include, inter alia, polyether siloxanes, particularly water-soluble block copolymers of siloxanes and polyethers. These compounds generally are prepared by joining a copolymer of ethylene oxide and propylene oxide or a homopolymer of ethylene oxide to a polydimethylsiloxane radical. Suitable stabilizers against the effects of aging and weathering and substances having fungistatic and bacteriostatic effect include, inter alia, phenolic and aromatic amine antioxidants, UV-stabilizers, hindered carbodiimides known to retard hydrolysis and oxidation, arsenic fungicidal compounds, tin and mercury bactericides, and the like.

Fillers which can be used for the purpose of extension or reinforcement of the polymeric elastomers and foams of the present invention include, inter alia, amorphous silicone hydroxides, carbon black, walnut and pecan shells, cork, cellulose, starch, calcium carbide, zinc oxide, titanium dioxide, clays, calcium wollastonite, and the like.

Continuing now with FIGS. 1A-2B, as referenced above, the thermal support member 120 also comprises one or more thermal elements 140. Such thermal element 140 can be at least partially, and more preferably fully, encased within the polymeric member 130. However, it should be understood that at least a portion of such thermal element 140 may necessarily protrude or extend outside the polymeric member 130 as an extended portion 142, such as for connecting to a controller or other operating device, without departing from the scope of the invention.

The purpose of the thermal element 140 is to provide a heating and/or cooling effect to the user. In some preferred embodiments, the thermal element 140 can have both heating and cooling capabilities. Preferably, the heat/energy transfer between the thermal element 140 and the user can be thermally conducted through the cushioning polymer 132 component of the thermal support member 120. In some embodiments, such heating and/or cooling effect can be accomplished through the use of energy (e.g., electricity). In some more preferred embodiments, such heating and/or cooling effect can be accomplished through the use of heated or cooled liquids and/or gases (e.g., air, water, steam, Freon, etc.) which can pass through the thermal element 140, in which case the thermal element 140 will typically have a substantially hollow structure (e.g., tubing, piping, HVAC ducting, etc.). However, it should be understood that other thermal transfer techniques known to persons having ordinary skill in the art (e.g., conduction, induction, radiation, etc.) can also be utilized without departing from the scope of the invention, in which case the thermal element 140 may or may not be substantially hollow. In some embodiments, the invention can utilize a combination of different types of thermal elements 140 without departing from the scope of the invention.

In one non-limiting example, the thermal element 140 can be flexible ¼-inch (6.4 mm) polyethylene (PEX) tubing, such as SHARKBITE SKU No. U850W50 PEX-B piping available from RWC, having a place of business located in Atlanta, Georgia, U.S.A. Other suitable thermal element 140 materials include, inter alia, plastics (e.g., PVC, polypropylene, styrene butadiene copolymers, etc.), rubber and metals (e.g., copper, steel, iron, chromium, nickel, titanium, zirconium, etc.), and combinations thereof, as well as other thermally conductive materials as would be known to persons having ordinary skill in the art.

Where the inventive decubitus prevention device 100 has the capability of inclining, declining or folding, it may be desirable to include flexible portions in the thermal element 140 at least at the bending point location(s) such that the decubitus prevention device 100 can conform while the thermal support member 120 remains fully functional.

With particular reference to FIG. 1A, in some preferred embodiments, the thermal support member 120 can include a thermal device component 200 which can be interconnected to the thermal element 140. The purpose of the thermal device component 200 is to provide heated and/or cooled fluids (i.e., liquids or gases) or energy (e.g., thermal energy) to the thermal element 140. Suitable thermal device components 200 include those known to persons having ordinary skill in the art, such as heat pumps, air conditioners, furnaces, boilers, heating elements, electrical connections, and the like.

In some embodiments, the thermal support member 120 and/or a thermal device component 200 can include a sensor (not shown) which can be interconnected to a controller (not shown). In addition, the thermal support member 120 and/or a thermal device 200 can include a user interface (not shown). For example, such a user interface can be located proximate to the thermal support member 120 or a thermal device 200, and/or such a user interface can be remotely located (e.g., via wireless connection). Suitable sensors, controllers and user interfaces include those known to persons having ordinary skill in the art, and can further include those described in U.S. Pat. No. 6,584,628 to Kummer et al., U.S. Pat. No. 7,480,953 to Romano et al., U.S. Pat. No. 7,931,607 to Biondo et al., U.S. Pat. No. 7,975,335 to O'Keefe et al., U.S. Pat. No. 8,108,957 to Richards et al., U.S. Pat. No. 8,572,778 to Newkirk et al., U.S. Pat. No. 8,856,992 to Lafleche et al., U.S. Pat. No. 8,973,186 to Bhai, U.S. Pat. No. 9,468,307 to Lafleche et al., U.S. Pat. No. 10,426,681 to Gibson et al., U.S. Pat. No. 10,827,844 to Mcknight et al., US Publication No. 2016/0250088 to Williamson et al. and WO 2016/167617 A1 entitled “Mattress for Massage”, each of which is incorporated herein by reference in a manner that is consistent herewith.

In some embodiments, the thermal support member 120 can optionally comprise a thermal element support component 144. As referenced above, the thermal element 140 can be at least partially encased in the polymeric member. Preferably, the length and width of the thermal element 140 will be substantially parallel (e.g., within a parallel plane) to the planar surface of the top side of the polymeric member 130 (and thus the top side 121 of the thermal support member 120) to help ensure a more even or uniform heating and/or cooling effect, though it need not be (such as when a non-uniform heating and/or cooling effect is desired). Depending on the flexibility and/or material of construction of the particular thermal element 140, it may pose a challenge to keep the thermal element substantially level (i.e., planarly parallel to the top side of the polymeric member 130) during the process of manufacturing the thermal support member 120. Thus, in some instances, it may be desirable to include one or more optional thermal element support components 144 to assist with keeping the thermal element at a designated height (or to have a designated contour) along its length and width during the manufacturing process. Preferably, any such thermal element support component 144 will be flexible such that it does not interfere with the function of the thermal support member 120. Suitable thermal element support components 144 include those known to persons having ordinary skill in the art, such as foams, string, tape, hook-and-loop, and the like, and combinations thereof. In one non-limiting example, a flexible thermoplastic foam (e.g., standard mattress foam) can have a length and width that are generally equivalent to that of a particular thermal element 140, and can have a predetermined height (and/or contour) at which the thermal element 140 is desired be disposed within the polymeric member 130. Accordingly, a support component(s) can be placed into, or affixed to, a mold (not shown). The thermal element 140 can then be disposed upon the top side of, or attached to, the thermal element support component(s) 144 while polymeric reaction media is being added to the mold. Alternatively, if the thermal element 140 happens to float in the reaction media, it may be desirable to fasten the thermal element 140 to the thermal element support components 144 (e.g., via stitching, tape, glue, hook-and-loop, etc.), or to alternatively place the thermal element support components 144 atop the thermal element 140.

In some embodiments, the thermal support member 120 can optionally comprise a bottom side barrier layer 136. Such optional bottom side barrier layer 136 will typically be disposed in a laid-flat configuration upon the planar bottom side of the polymeric member 130 (and thus the bottom side 122 of the thermal support member 120), thus forming a laminated-type of structure. The purpose of the optional bottom side barrier layer 136 includes, inter alia, preventing direct contact between the polymeric member 130 and the top side 151 of the massage member 150 and/or preventing potential leakage of plasticizer (if any) which could occur from the cushioning polymer 132 component of the polymeric member 130. Suitable bottom side barrier layers 136 can desirably be in the form of a substrate, and can comprise materials known to persons having ordinary skill in the art, for example plastics (e.g., polyethylene, polypropylene, PVC, etc.), polyester, elastic webbing, Spandex, vinyl, thermoplastic foam, thermoset foam, natural and synthetic leather, Gortex, nonwovens (e.g., meltblown, spunbond, etc.), coated woven textiles, Teflon®, and the like, and combinations thereof. The optional bottom side barrier layer 136 can comprise any functional thickness, but will typically be relatively thin, such as between about 0.1 mm and about 1 mm. However, it should be understood that an optional bottom side barrier layer 136 can have a thickness of less than 0.1 mm or greater than 1 mm without departing from the scope of the invention. Typically, an optional bottom side barrier layer 136 can be adhered to the bottom side 122 of the thermal support member 120 by virtue of being placed in contact with the reaction media (i.e., prior to fully curing) which forms the cushioning polymer 132 component of the polymeric member 130 during production. Alternatively, such an optional bottom side barrier layer 136 can be affixed to the polymeric member 130 post-production using attachment means known to persons having ordinary skill in the art (e.g., stitching, adhesives, mechanical fasteners, etc.) without departing from the scope of the invention.

In some embodiments, the thermal support member 120 can optionally comprise a top side barrier layer 138. Such optional top side barrier layer 138 will typically be disposed in a laid-flat configuration upon the generally planar top side of the polymeric member 130 (and thus the top side 121 of the thermal support member 120), thus forming a laminated-type structure. The purpose of the optional top side barrier layer 138 includes, inter alia, preventing direct contact with the polymeric member 130, providing an aesthetically desirable feel to the top side 121 of the thermal support member 120 and/or preventing potential leakage of plasticizer (if any) which may occur from the cushioning polymer 132 component of the polymeric member 130. Suitable top side barrier layers 138 can desirably be in the form of a substrate or pad, and can comprise materials known to the persons having ordinary skill in the art, for example plastics (e.g., polyethylene, polypropylene, PVC, etc.), polyester, elastic webbing, Spandex, vinyl, thermoplastic foam, thermoset foam, natural and synthetic leather, Gortex, nonwovens (e.g., meltblown, spunbond, etc.), coated woven textiles, cotton padding, wool padding, felt, and the like, and combinations thereof. The optional top side barrier layer 138 can comprise any functional thickness, but will typically be relatively thin, such as between about 0.1 mm and about 10 mm. However, it should be understood that an optional top side barrier layer 138 can have a thickness of less than 0.1 mm or greater than 10 mm without departing from the scope of the invention. Typically, an optional top side barrier layer 138 can be adhered to the top side 121 of the thermal support member 120 by virtue of being placed in contact with the reaction media (i.e., prior to fully curing) which forms the cushioning polymer 132 of the polymeric member 130 during production. Alternatively, such an optional top side barrier layer 138 can be affixed to the polymeric member 130 post-production using attachment means known to persons having ordinary skill in the art (e.g., stitching, adhesives, mechanical fasteners, etc.) without departing from the scope of the invention.

As referenced above, in some embodiments, the thermal support member 120 can optionally comprise a sheath member 110 which can be disposed upon and/or can at least partially surround or encase the thermal support member 120 (see e.g. FIG. 1A). Such flexible sheath member 110 may be desirable for numerous reasons including, inter alia, visual aesthetics, surface feel, additional structural support, plasticizer leakage barrier, etc. Suitable sheath members 110 can comprise materials known to the persons having ordinary skill in the art, such as plastics (e.g., polyethylene, polypropylene, PVC, etc.), polyester, elastic webbing, Damask, Spandex, satin, Sateen, vinyl, thermoplastic foam, thermoset foam, Gortex, nonwovens (e.g., meltblown, spunbond, etc.), coated woven textiles, cotton, wool, felt, natural and synthetic leather, natural and synthetic rubbers, and the like, and combinations thereof. Preferably, such sheath member 110 will have a flexibility that is at least equivalent to, or greater than, that of the thermal support member 120, though it need not be. The optional sheath member 110 can comprise any functional thickness, but will typically be relatively thin, such as between about 0.5 mm and about 10 mm. However, it should be understood that an optional sheath member 110 can have a thickness of less than 0.5 mm or greater than 10 mm without departing from the scope of the invention.

In some embodiments, the thermal support member 120 can optionally comprise a flexible or semi-rigid horizontal bottom support member (not shown) which can be disposed at least partially upon the bottom side 122 of the thermal support member 120. Such horizontal bottom support member may be desirable to provide additional support to the polymeric member 130 and/or to provide variable support (e.g., firmness variation) to particular locations of a user's body. Suitable bottom support members include those known to persons having ordinary skill in the art, such as substrates, cushions, pads, air bladders, rods, rails, blocks, and the like.

In some embodiments, the thermal support member 120 can optionally comprise a flexible, semi-rigid or rigid vertical side support member (not shown) which can be disposed at least partially along one or more of the vertical side 123,124,125,126 portions of the thermal support member 120 (i.e., at least partially around the outer perimeter of the thermal support member 120 and/or at least partially along the z-axis 3). Such vertical side support member(s) may be desirable to provide additional confining support to the polymeric member 130. Such side support members include those known to persons having ordinary skill in the art and can comprise materials including, but not limited to, plastics, fiberglass, wood, metal, and the like.

The invention also includes a method for making a thermal support member 120. One non-limiting exemplary method can include:
A. Providing a suitable mold (not shown) optionally comprising a suitably sized opening through which an extended portion 142 of a thermal element 140 can closely (securely) fit;
B. Placing the mold onto a suitable surface;
C. Optionally aligning and placing a bottom side barrier layer 136 into the mold in a laid-flat configuration upon the surface;
D. Optionally aligning and placing a vertical side support member at an inner perimeter location within the mold;
E. Optionally aligning and placing a thermal element support component 144 into the mold or upon the top side of the optional bottom side barrier layer 136;
F. Aligning and placing a thermal element 140 within the mold and/or upon the top side surface of the optional thermal element support component 144;
G. Adjusting the position of the thermal element 140 and optional thermal element support component 144 such that the extended portion 142 of a thermal element 140 extends through the designated opening(s) in the mold;
H. Preparing a suitable liquid polymeric reaction media;
I. Disposing a quantity of liquid reaction media into the mold, preferably such that the reaction media substantially covers (i.e. envelops) the thermal element 140;
J. Optionally aligning and placing a top side barrier layer 138 in a laid-flat configuration upon the planar top side of the liquid reaction media;
K. Allowing the reaction media to fully cure to form a polymeric member 130; and
L. Removing the combination (i.e., polymeric member 130 and thermal element 140, as well as optional thermal element support component 144 if present, optional bottom side barrier layer 136 if present, optional vertical side support member if present and optional top side barrier layer 138 if present) to form an inventive thermal support member 120 of the present disclosure.

It should be understood that as an alternative to optional step C, optional step D, and/or optional step J, a bottom side barrier layer 136, vertical side support member and/or top side barrier layer 138 can be added (if desired) to the thermal support member 120 post-production thereof. In some embodiments, the method can optionally include applying (pre- or post-production) a sheath member 110 to the thermal support member 120 to at least partially encase the thermal support member 120. Furthermore, the method can additionally, or alternatively, comprise a step of disposing (pre- or post-production) a horizontal bottom support member upon the planar bottom side 122 of the thermal support member 120.

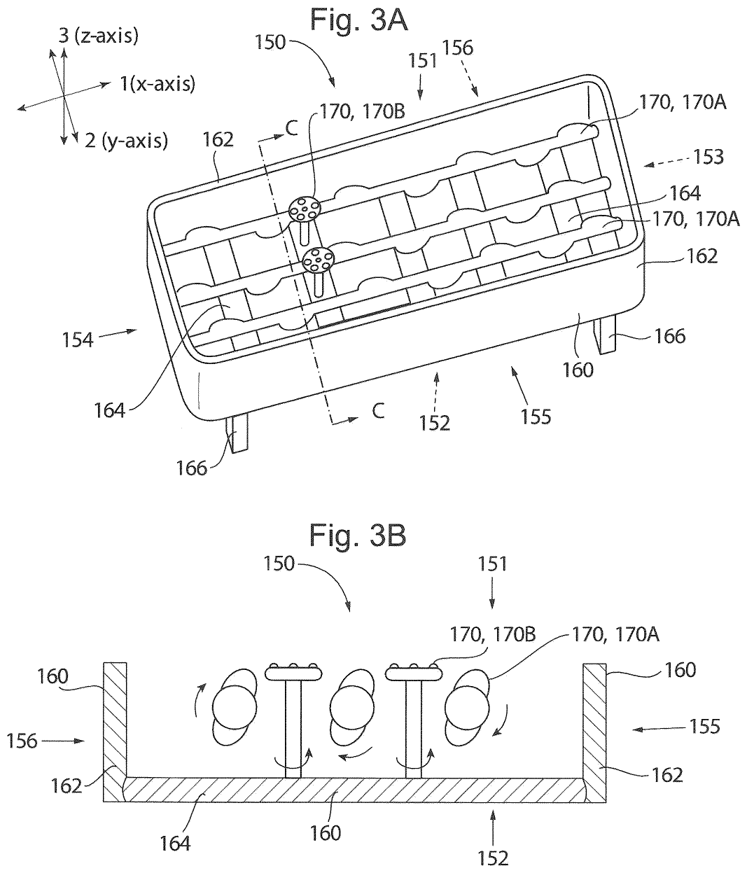

Returning now to FIGS. 1A-1B, and with additional reference to FIGS. 3A-3B, the inventive decubitus prevention device 100 also comprises a massage member 150. The purpose of the massage member 150 is to provide at least a slight, and more preferably a significant, physical manipulation of, or impact to, at least a portion of the body of a user. Accordingly, the massage member 150 comprises one or more manipulation elements 170 which indirectly contact the user through the thermal support member 120.

The massage member 150 is typically disposed beneath the thermal support member 120 such that the manipulation elements 170 are in contact with, and impress into, the bottom side 122 of the thermal support member 120. In some preferred embodiments, the massage member 150 comprises a generally horizontal first or top side 151, an opposing generally horizontal second or bottom side 152 distal to the first side 151, a vertical third or “head” generally planar side or end 153 disposed orthogonally between the first side 151 and the second side 152, an opposing vertical fourth or “foot” generally planar side or end 154 distal to the third side 153, a vertical fifth generally planar side 155 disposed orthogonally between the first side 151 and the second side 152 and between the third side 153 and the fourth side 154, and an opposing vertical sixth generally planar side 156 distal to the fifth side 155. In some embodiments, the massage member 150 can have outer dimensions in the x-y plane that are substantially equivalent to the dimensions in the x-y plane of the thermal support member 120 (and thus of the decubitus prevention device 100). In other embodiments, the length and width of the housing component 160 of the massage member 150 can be greater than the length and width of the thermal support member 120 such that the bottom side 122 of the thermal support member 120 rests solely upon the manipulation elements 170 disposed within the massage member 150. It should be understood that the massage member 150 can have dimensions along the x-axis 1 and/or the y-axis 2 which are less than, equivalent to, or greater than, the dimensions in the x-y plane of the thermal support member 120 without departing from the scope of the invention. In addition, the massage member 150 can be present as a single component, or can be present as a plurality of separate components located in particular positions adjacent to the bottom side 122 of the thermal support member 120. For purposes of brevity, a massage member 150 in the form of a unitary component will be described herein.

In some preferred embodiments, the massage member 150 can comprise a framework or housing component 160 wherein one or more manipulation elements 170 can be substantially disposed within an interior portion of such housing component 160. Accordingly, the housing component 160 will typically comprise a rigid or semi-rigid material, although flexible materials can also be suitable in some embodiments without departing from the scope of the invention. Suitable materials for use in the housing component 160 include those known to persons having ordinary skill in the art, such as metals, wood, plastics, fiberglass, and the like, and combinations thereof. In addition, the housing component 160 may be present as a single unit, or may comprise a composite of various sections. For example, in one non-limiting exemplary embodiment, the housing component 160 can comprise an outer frame element 162 and interconnecting cross member elements 164 which can help provide structural support and/or can provide mounting locations for one or more manipulation elements 170. In some preferred embodiments, the housing component 160 can optionally comprise elevation elements 166 (e.g., legs) which can assist with setting the inventive decubitus prevention device 100 at a particular height (such as measured from the floor, for example). Such optional elevation elements 166 can be static or height-adjustable without departing from the scope of the invention. Suitable elevation elements 166 include those known to persons having ordinary skill in the art, including extensions, rails, pegs, blocks, wheels, rollers, and the like, and combinations thereof.

As referenced above, the massage member 150 also comprises one or more manipulation elements 170. The purpose of a manipulation element 170 is to apply an impactful force through the thermal support member 120 and upon at least a portion of a user's body. In some preferred embodiments, a plurality of such forces will be intermittently applied to provide a massage effect or other such therapeutic effect to at least a portion of a user's body. Such forces can vary by area, such as to provide greater forces in one area and comparatively lesser forces in another area, though it need not be without departing from the scope of the invention. In some embodiments, such differentiation of forces (if desired) can be accomplished by utilizing a combination of different size or different types of manipulation elements 170, although the application of varying forces can also be accomplished using the same type and size of manipulation elements 170 in other embodiments. In one non-limiting example, a massage member 150 can comprise a housing component 160 having a frame element 162 and a plurality of cross member elements 164, and can further comprise a combination of differing manipulation elements 170 including a first type of manipulation element 170A (e.g., in the form of axially rotating camshafts) and a second type of manipulation element 170B (e.g., in the form of roller balls mounted on rotational wheels) (see e.g., FIGS. 3A-3B). In this illustrated non-limiting example, a plurality of the first type of manipulation elements 170A can extend along substantially the length (x-axis 1) of the massage member 150 within the housing component 160 and can be spaced-out along the width (y-axis 2) of the massage member 150. In addition, a plurality of the second type of manipulation elements 170B can be substantially aligned across the width (y-axis 2) of the housing component 160 generally at the location of a user's lumber/buttocks region, for example. Where the outer edges of the bottom side 122 of the thermal support member 120 are intended to rest upon the housing component 160, the height (along the z-axis 3) of the manipulation elements 170 can preferably be greater than the height of the housing component 160 to help ensure the manipulation elements 170 can sufficiently impress into the thermal support member 120 to produce impactful forces which are detectable by a user disposed upon the top side 121 of the thermal support member 120. In contrast, where the thermal support member 120 is sized to fit into the housing component 160 and substantially rest upon the manipulation elements 170, such issue is of less concern.

Any suitable manipulation element 170 can be utilized for the massage member 150, including those known to persons having ordinary skill in the art, such as rollers, balls, knobs, cones, ridges, pistons, plungers, cams, augers, bladders, vibration elements, and the like, and combinations thereof. Such manipulation elements 170 are disclosed and described in the aforementioned U.S. Provisional Application No. 63/306,490 entitled “Decubitus Prevention Device” to Kriesel et al., which is incorporated herein by reference in its entirety. Other suitable manipulation elements 170 can include those described in U.S. Pat. No. 5,303,436 to Dinsmoor, III et al., U.S. Pat. No. 6,584,628 to Kummer et al., U.S. Pat. No. 7,260,860 to Chambers et al., U.S. Pat. No. 7,716,766 to Poulos, U.S. Pat. No. 7,914,471 to Chen, U.S. Pat. No. 7,937,791 to Meyer et al., U.S. Pat. No. 8,683,633 to Cao, U.S. Pat. No. 8,910,334 to Lafleche et al., U.S. Pat. No. 9,204,732 to Wyatt et al., WO 2018/033114 A1 entitled “Massage Mattress”, Chinese Patent No. CN 109512597 A entitled “A Kind of Multifunctional Ball Massage Mattress”, Chinese Patent No. CN 208355066 U entitled “A Kind of Massage Mattress”, Chinese Patent No. CN 210870679 U entitled “Multifunctional Massage Mattress”, South Korean Patent No. KR20140088748A entitled “Massage Chair”, South Korean Patent No. KR20170098740A entitled “Massage Chair with Rollers for Back and Thigh”, South Korean Patent No. KR20170122526A entitled “A Massage Chair”, and South Korean Patent No. KR20170123803A entitled “Massage Mattress”, each of which is incorporated herein by reference in a manner that is consistent herewith.

Such manipulation elements 170 can be operated via means known to persons having ordinary skill in the art, such as motors, servos, pumps, vacuums, and the like, and combinations thereof. In some embodiments, a plurality of manipulation elements 170 can be interconnected via drive shafts, belts, and/or other such means known to persons having ordinary skill in the art. Where the inventive decubitus prevention device 100 is capable of inclining, declining, folding or otherwise bending, it may be desirable to include hinges upon the housing component 160 and couplings or other suitable means as known to persons having ordinary skill in the art upon the operation mechanisms of the manipulation elements 170 such that the decubitus prevention device 100 can conform to such bending portions while remaining fully functional.

The thickness (i.e., height as measured along the z-axis 3) of the massage member 150 is not limited to any particular measurement, and will be at least partly dependent upon numerous factors including, inter alia, the dimensions of the housing or framework 160, the dimensions of the manipulation element(s) 170, the type of manipulation element(s) 170, the desired amount of impression by a manipulation element 170 into the bottom side 122 of the thermal support member 120, the desired clearance between the bottom side 152 of the massage member and the bottom side of a particular manipulation element 170, etc. In some embodiments, the thickness or height of the massage member 150 will be such that the housing or framework 160 is at least substantially equivalent to, or less than, the height of the manipulation element 170 disposed therein, though it need not be without departing from the scope of the invention. In general, the overall height of the massage member 150 (not including any optional elevation elements 166) can range between 10 cm to about 40 cm. However, it should be understood that the massage member 150 can have a height of less than 10 cm or greater than 40 cm without departing from the scope of the invention.

As referenced above, in some embodiments, the massage member 150 can optionally comprise a sheath member 110 which can be disposed upon and/or can at least partially surround or encase the massage member 150. Such flexible sheath member 110 may be desirable for numerous reasons including, inter alia, visual aesthetics, additional structural support, protection of the bottom side 152 of the massage member 150, protection of the manipulation elements 170 from potential plasticizer leakage (if any) from the thermal support member 120, etc. Suitable sheath members 110 can comprise materials known to the persons having ordinary skill in the art, such as plastics (e.g., polyethylene, polypropylene, PVC, etc.), Teflon®, polyester, elastic webbing, Damask, Spandex, satin, Sateen, vinyl, Gortex, nonwovens (e.g., meltblown, spunbond, etc.), coated woven textiles, cotton, wool, felt, natural and synthetic leather, natural and synthetic rubbers, and the like, and combinations thereof. Preferably, such sheath member 110 will have a flexibility that does not diminish the impact of a manipulation element 170. The optional sheath member 110 can comprise any functional thickness, but will typically be relatively thin, such as between about 0.5 mm and about 10 mm. However, it should be understood that an optional sheath member 110 can have a thickness of less than 0.5 mm or greater than 10 mm without departing from the scope of the invention.

The invention of the present disclosure also includes a first method of making an inventive decubitus prevention device 100. In this embodiment, the method can include:
A. Providing a thermal support member 120 as described above;
B. Providing a massage member 150 as described above;
C. Disposing the bottom side 122 of the thermal support member 120 upon the top side 151 of the massage member 150 to form an inventive decubitus prevention device 100 of the present disclosure; and
D. Optionally at least partially encasing the decubitus prevention device 100 in a sheath member 110.

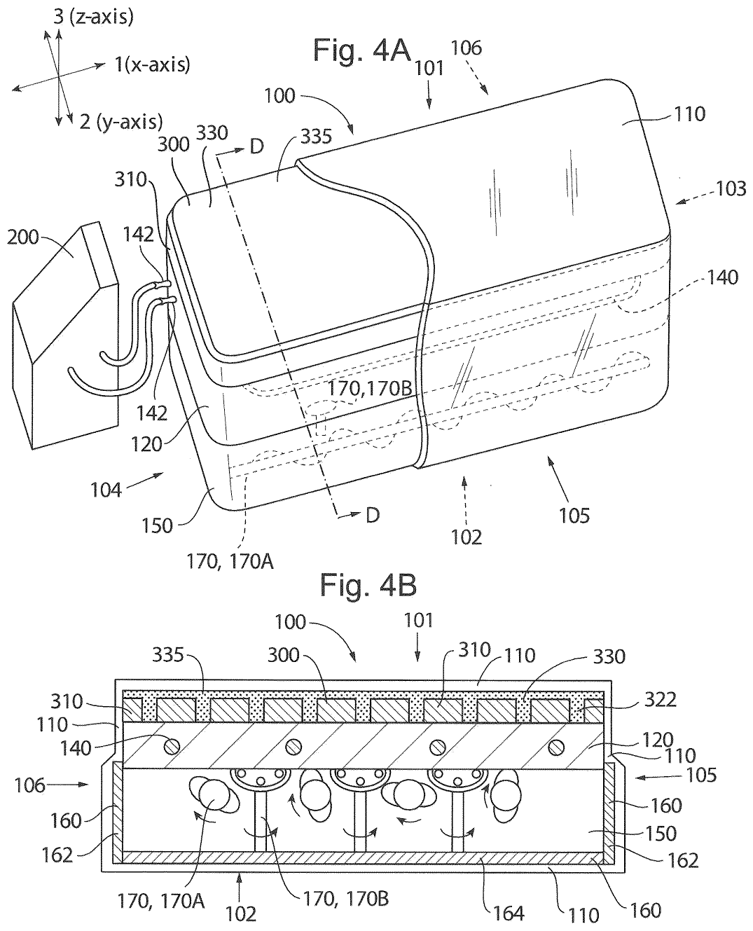

Referring now to FIGS. 4A-4B, another embodiment of the inventive decubitus prevention device 100 comprises a thermal support member 120 (as described above) disposed upon the top side 151 of a massage member 150 (as described above), and further comprises a therapeutic member 300 disposed upon the top side 121 of the thermal support member 120. In some aspects of this embodiment, the inventive decubitus prevention device 100 can additionally comprise an optional sheath member 110 (e.g., a mattress covering, etc.) which can at least partially cover or enclose the therapeutic member 300, the thermal support member 120 and/or the massage member 150. Such sheath member 110 can, inter alia, provide an aesthetically pleasing surface feel, provide an aesthetically pleasing visual appearance, help prevent potential leakage of plasticizer (if any) from the polymeric member 130 and/or the therapeutic member 300, reduce the level of any sound emitted by the device 100, assist with maintaining the positions of the therapeutic member 300, the thermal support member 120 and the massage member 150 with respect to each other, and present the device 100 as a single unit, for example. Suitable materials for use as a sheath member 110 include those known to persons having ordinary skill in the art, such as plastics (e.g., polyethylene, polypropylene, PVC, etc.), polyester, elastic webbing, Damask, Spandex, satin, Sateen, vinyl, thermoplastic foam, thermoset foam, Gortex, nonwovens (e.g., meltblown, spunbond, etc.), coated or uncoated woven textiles, cotton, wool, felt, natural and synthetic leather, natural and synthetic rubbers, and the like, and combinations thereof. Preferably, such sheath member 110 will have a flexibility that is at least equivalent to, or greater than, that of the therapeutic member 300 component of the inventive decubitus prevention device 100 of this embodiment, though it need not be. The optional sheath member 110 can comprise any functional thickness, but will typically be relatively thin, such as between about 0.5 mm and about 10 mm. However, it should be understood that an optional sheath member 110 can have a thickness of less than 0.5 mm or greater than 10 mm without departing from the scope of the invention.

In some aspects of this embodiment, the inventive decubitus prevention device 100 can also comprise additional layers (e.g., nonwoven layers, foam layers, felt layers, etc.) (not shown) which may be disposed atop the therapeutic member 300, beneath the massage member 150, between the therapeutic member 300 and the thermal support member 120, between the thermal support member 120 and the massage member 150, and/or along the side portions of the therapeutic member 300, the thermal support member 120 and/or the massage member 150, without departing from the scope of the invention.

The purpose of the therapeutic member 300 is to provide additional comfort to the user, to provide a therapeutic effect to the user, to more uniformly distribute the heating effect and/or or cooling effect provided by the thermal support member 120, and/or to distribute or absorb some of the impactful forces provided by the massage member 150. In some aspects, the therapeutic member 300 can additionally transfer heat and moisture away from the user, notwithstanding the presence of the thermal support member 120.

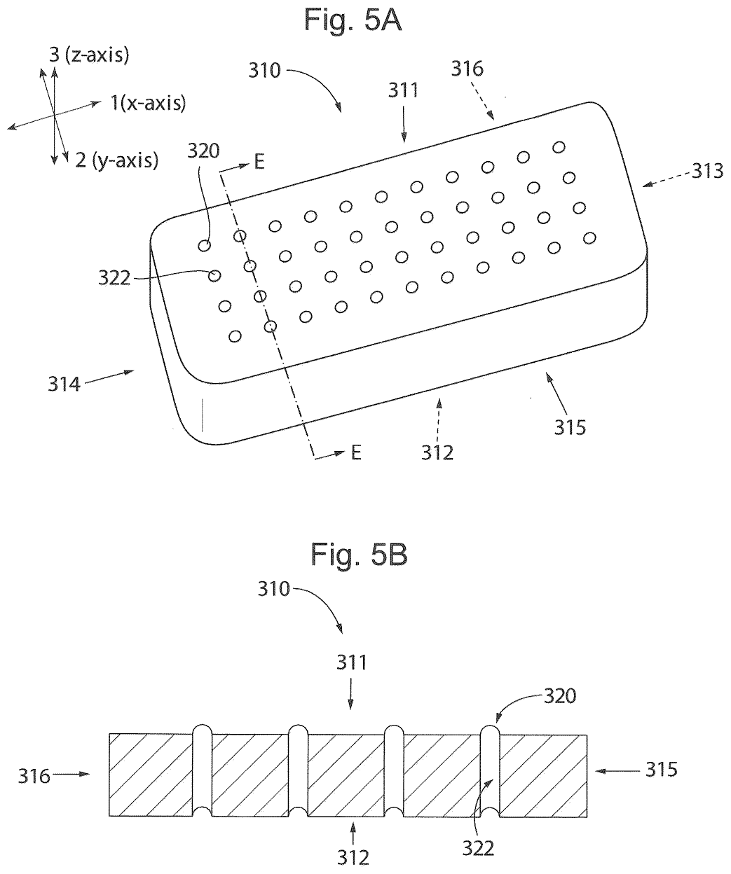

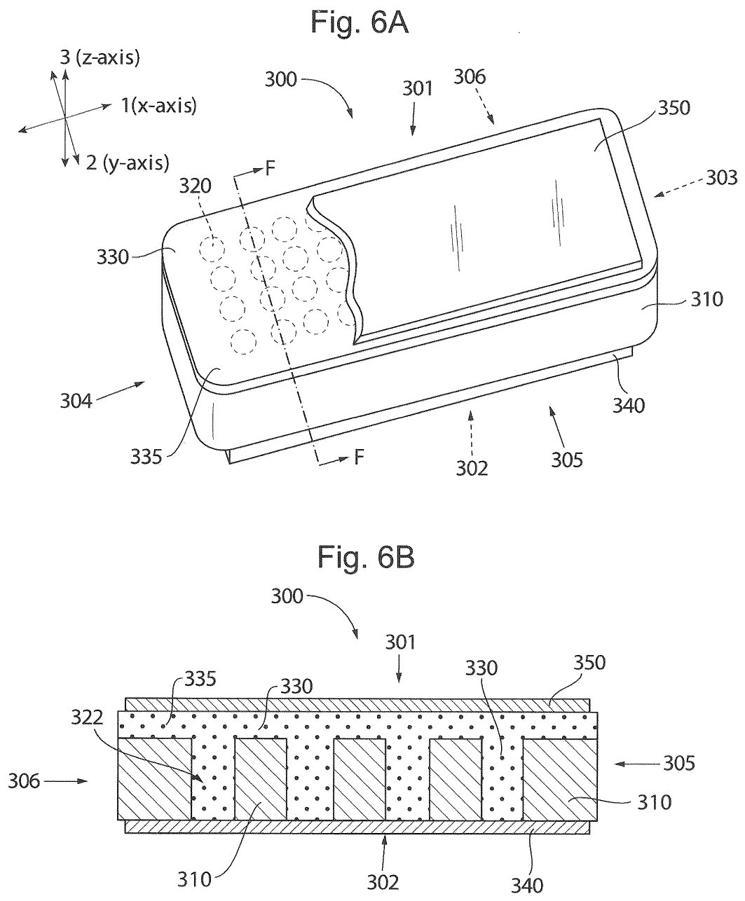

As illustrated in the non-limiting examples shown in FIGS. 4A-4B, and with additional reference to FIGS. 5A-6B, the therapeutic member 300 can comprise a generally horizontal first or top major planar side 301, an opposing generally horizontal second or bottom major planar side 302 distal to the first side 301, a vertical third or “head” generally planar side, end or edge 303 disposed orthogonally between the first side 301 and the second side 302, an opposing vertical fourth or “foot” generally planar side, end or edge 304 distal to the third end 303, a vertical fifth generally planar side or edge 305 disposed orthogonally between the first side 301 and the second side 302 and between the third end 303 and the fourth end 304, and an opposing vertical sixth generally planar side or edge 306 distal to the fifth side 305.

In some preferred aspects of this embodiment, the therapeutic member 300 can have dimensions in the x-y plane that are substantially equivalent to the dimensions in the x-y plane of the thermal support member 120. However, it should be understood that the therapeutic member 300 can have dimensions along the x-axis 1 (i.e., length) and/or the y-axis 2 (i.e., width) which are less than, or greater than, the dimensions in the x-y plane of the thermal support member 120 without departing from the scope of the invention. In addition, the top side 301 of the therapeutic member 300 may have a generally flat surface, or may comprise contours or other three-dimensional (i.e., non-flat) surface characteristics without departing from the scope of the invention. Further, the height or thickness (i.e., as measured along the z-axis 3) of the therapeutic member 300 can be generally uniform or variable. For example, the therapeutic member 300 can have a greater thickness in one or more particular locations (e.g., lumbar support, head support, etc.) without departing from the scope of the invention. In general, the therapeutic member 300 can have an average thickness (i.e., as measured along the z-axis 3) of about 1 cm to about 40 cm, such as about 1.5 cm to about 30 cm, or about 2 cm to about 20 cm. However, it should be understood that the therapeutic member 300 can have an average thickness of less than 1 cm or greater than 30 cm without departing from the scope of the invention.

Referring now to FIGS. 5A-6B, in one non-limiting example of the invention of this embodiment, a therapeutic member 300 comprises a mitigation member 310 and a thermal-conductive polymer 330, wherein the thermal-conductive polymer 330 is disposed at least partially through the mitigation member 310, generally along the z-axis 3. Typically, the thermal-conductive polymer 330 will be applied to the mitigation member 310 while in a liquid (i.e., uncured or partially-cured) reaction media form. In some embodiments, the thermal-conductive polymer 330 can be disposed into the mitigation member 310 (e.g., generally in the z-direction 3) by virtue of the reaction media soaking into the mitigation member 310 (such as migrating into and/or through the open cells of a foam, for example). More preferably, the mitigation member 310 can comprise a plurality of perforations or apertures 320 disposed at least partially through the mitigation member 310 (typically in the general z-direction 3) to provide channels 322 for the reaction media to flow.

With particular reference to FIGS. 5A-5B, the mitigation member 310 can comprise a generally horizontal first or top major planar side 311, an opposing generally horizontal second or bottom major planar side 312 distal to the first side 311, a generally vertical third or “head” side, end or edge 313 disposed orthogonally between the first side 311 and the second side 312, an opposing generally vertical fourth or “foot” side, end or edge 314 distal to the third end 313, a generally vertical fifth side or edge 315 disposed orthogonally between the first side 311 and the second side 312 and between the third end 313 and the fourth end 314, and an opposing generally vertical sixth side or edge 316 distal to the fifth side 315. In some aspects of this embodiment where the thermal-conductive polymer 330 completely soaks into the top side 311 of the mitigation member 310 (i.e., the polymer 330 does not form a layer atop the mitigation member 310), the dimensions of the mitigation member 310 can substantially define the length (as measured along the x-axis 1), width (as measured along the y-axis 2) and height (as measured along the z-axis 3) of the therapeutic member 300. However, in other aspects where the thermal-conductive polymer 330 forms a layer upon the top side 311 of the mitigation member 310 (in addition to optionally soaking into the mitigation member 310), the height of the therapeutic member 300 may be greater than the height of the mitigation member 310 alone. In addition, depending on the type of material used to construct the mitigation member 310, the presence of the thermal-conductive polymer 330 may result in an expansion, and/or deformation (e.g., bowing out) of the sides 313,314,315,316, of the mitigation member 310 in the length and/or width dimensions.

The top side 311 of the mitigation member 310 may have a generally flat surface, or may comprise contours or other three-dimensional (i.e., non-flat) surface characteristics without departing from the scope of the invention. Furthermore, the height or thickness (i.e., as measured along the z-axis 3) of the mitigation member 310 can be generally uniform or variable. For example, the mitigation member 310 can have a greater thickness in one or more particular locations (e.g., lumbar support, head support, etc.), or may have a dimpled or other patterned topography, without departing from the scope of the invention. In general, the mitigation member 310 can have an average thickness of about 1 cm to about 30 cm, such as about 1.5 cm to about 20 cm, or about 2 cm to about 10 cm.

The mitigation member 310 can comprise any material that provides a desired softness, resiliency, flexibility, absorbency and/or cushioning effect. Typically, the mitigation member 310 will comprise a relatively soft, flexible and resilient foam, such as a standard mattress foam. In some preferred aspects, the mitigation member 310 is also absorbent. For example, a foam substrate suitable for use as a mitigation member 310 includes 4200-245848 (a 1.5 pound per cubic foot/17 ILD, open cell polyether foam), available from American Converters, having a place of business located in Fridley, Minnesota, U.S.A.

By way of a non-limiting example only, a suitable foam can include an elastomeric thermoplastic foam. In general, thermoplastic foams have a cellular structure, with cells defined by cell membranes and struts. The struts are formed at the intersection of cell membranes, with the cell membranes covering interconnecting cellular windows between the struts. Foams may further contain cell orifices within the membranes that can provide doorways into adjoining cells. Accordingly, the foam may define a plurality of open cells and/or closed cells which are separated from one another by cell membranes and struts. Cell sizes may be in the range of about 10 microns to about 1000 microns as measured by ASTM D3576. In some aspects, a “fine” foam can have foam cell sizes in the range of about 10 microns to about 500 microns, such as about 20 microns to about 300 microns. In other aspects, a “coarse” foam can have foam cell sizes in the range of about 500 microns to about 1000 microns. The specific number and size of cells can be determined by the foam formulation, as well as the processing parameters selected.

In general, a foam having a low density and low bending modulus can provide enhanced softness and flexibility. A thermoplastic elastomer can also be added to enhance softness, flexibility and elasticity. In addition, a foam can be formulated and processed to exhibit a low compression set.

Suitable foams may be substantially closed-celled, substantially open-celled, or a combination thereof. In some aspects, the foam can have an open cell structure of about 25% or greater, such as about 50% or greater, or 75% or greater, as measured by using a gas pycnometer according to ASTM D2856, Method C. In other aspects, the foam can have a closed-cell content of at least about 25%, such as at least about 50% or at least about 75%, which can help improve resiliency and/or compression resistance.

A thermoplastic foam can also have desirable basis weights. For example, in some aspects, the foam can have a basis weight of about 300 gsm or less. A thermoplastic foam can also have desirable densities. For example, the foam can have a density in the range of about 0.01 g/cc to about 0.5 g/cc or greater. Furthermore, densification of the foam at some point after the formation process can be employed to enhance functionality for specific applications.

A non-limiting exemplary thermoplastic foam can be made of at least one polymer that can be heated, formed and cooled repeatedly. The starting material used in the foam formulation can include at least one suitable base resin which could include a single thermoplastic polymer, a blend of thermoplastic polymers, or a blend of thermoplastic and non-thermoplastic polymers. Examples of base resins suitable for use in the foam formulation include styrene polymers, such as polystyrene or polystyrene copolymers or other alkenyl aromatic polymers, polyolefins including homo or copolymers of olefins, such as polyethylene, polypropylene, polybutylene, etc., polyesters, such as polyalkylene terephthalate, and combinations thereof. For example, in some aspects, a suitable base resin includes STYRON 685D polystyrene resin available from Dow Chemical Company, having a place of business located in Freeport, Texas, U.S.A.

Coagents and compatibilizers can also be utilized for blending such resins. Additionally, crosslinking agents can also be employed to enhance mechanical properties, foamability, and expansion. Such crosslinking may be accomplished by utilizing several means, including the use of electron beams or by chemical crosslinking agents such as organic peroxides.

It is suitable to utilize base resins which provide effective foamability, softness and flexibility. In general, resins having branched polymer chains tend to be more foamable. As such, flexibility, softness, and foamability can be manipulated by utilizing several means, including the use of polymer side groups, the incorporation of chains within the polymer structure to prevent polymer crystallization, the lowering of the glass transition temperature, the lowering of a given polymer's molecular weight distribution, the adjusting of melt flow strength and viscous/elastic properties including elongational viscosity of the polymer melt, the use of block copolymerization, the blending of polymers, the use of polyolefin homopolymers and copolymers including low (such as linear low), medium and high-density polyethylene and polypropylene which are normally made using Ziegler-Natta or Phillips catalysts and are relatively linear as well as those that can be engineered with elastic and crystalline areas, the use of syndiotactic, atactic and isotactic polypropylenes including those made using metallocene-based catalysts as well as blends of such and other polymers, and the use of olefin elastomers.

In some applications, it is suitable to utilize resins which provide foam composites that are soft and/or extensibly elastic. Softness and extensibility can be manipulated using several means, including the use of ethylene and α-olefin copolymers, particularly those made using either Ziegler-Natta or a metallocene catalyst such as metallocene catalyzed polyolefins, the use of polyethylene cross-linked with α-olefins and various ethylene ionomer resins, and the use of ethyl-vinyl acetate copolymers with other polyolefin-type resins.

Common modifiers for various polymers can also be reacted with chain groups to obtain suitable functionality. This includes the use of alkenyl aromatic polymers and ionomer resins. Suitable alkenyl aromatic polymers include alkenyl aromatic homopolymers and copolymers of alkenyl aromatic compounds and copolymerizable ethylenically unsaturated comonomers including minor proportions of non-alkenyl aromatic polymers and blends thereof.

Thermoplastic base resins could also contain blends of other polymers with the thermoplastic polymers, such as natural and synthetic organic polymers including cellulosic polymers, methyl cellulose, polylactic acids, polyvinyl acids, polyacrylates, polycarbonates, starch-based polymers, polyetherimides, polyamides, polymethylmethacrylates, and copolymer/polymer blends.

In some aspects, the foam formulation could include a polyurethane base resin, such as hydrophilic urethane prepolymer. Examples of suitable hydrophilic urethane prepolymers include isocyanate terminated or capped polyoxyalkylene ethers including polyoxyethylene polyol prepolymers. Other examples of suitable prepolymers are described in U.S. Pat. No. 4,137,200 to Woods et al., U.S. Pat. No. 4,209,605 to Hoy et al., U.S. Pat. No. 2,993,013 to Wolfe, Jr., and U.S. Pat. No. 3,805,532 to Kistner, each of which is incorporated herein by reference in a manner that is consistent herewith. General procedures for the preparation of such prepolymers are described by J. H. Saunders and X. C. Frisch in Polyurethanes Chemistry and Technology, Interscience Publishers, John Wiley & Sons, New York, Vol. XVI, Part 2, High Polymer Series, published in 1987, “Foam Systems” pages 7-26, and “Procedures for the Preparation of Prepolymers” pages 26 et seq., each of which is incorporated herein by reference in a manner that is consistent herewith.

In some aspects, the foam formulation can comprise toluene diisocyanate (TDI) base resin that is terminated with polyethylene polyol with less than 6% of the available unreacted NCO groups and a component functionality of 2 or less, such as TREPOL available from Rynel Ltd., Inc., having a place of business located in Boothbay, Maine, USA. In other aspects, the base resin can include HYPOL 2000/3000 grade prepolymers, available from Dow Chemical Co. which are water-activated polymeric liquid polyurethanes based on TDI. In general, a hydrophilic prepolymer is activated by the aqueous phase for polymerization upon mixing.

In addition to the base resin polymers discussed above, the foam formulation can also include at least one thermoplastic elastomer. For example, in some aspects, the foam formulation can comprise up to about 95-percent base resin by weight of the foam formulation (wt %), such as about 50 wt % to about 95 wt %, or about 50 wt % to about 80 wt % base resin and at least about 5 wt % thermoplastic elastomer, such as about 5 wt % to about 50 wt %, or about 20 wt % to about 50 wt % thermoplastic elastomer. In some aspects, the foam formulation can comprise substantially equal amounts of base resin and thermoplastic elastomer.

Suitable thermoplastic elastomers include, but are not limited to, rubbers, including natural rubber, styrene-butadiene rubber (SBR), polybutadiene, ethylene propylene terpolymers, and vulcanized rubbers including TPVs, rubber-modified polymers such as styrene elastomers, ethylene elastomers, butadiene, polybutylene resins, diblock, triblock, tetrablock, or other multi-block thermoplastic elastomeric and/or flexible copolymers such as polyolefin-based thermoplastic elastomers including random block copolymers including ethylene α-olefin copolymers, block copolymers including hydrogenated butadiene-isoprene-butadiene block copolymers, stereoblock polypropylenes, graft copolymers including ethylene-propylene-diene terpolymer or ethylene-propylene-diene monomer (EPDM), ethylene-propylene random copolymers (EPM), ethylene propylene rubbers (EPR), ethylene vinyl acetate (EVA), and ethylene-methyl acrylate (EMA), and styrenic block copolymers including diblock and triblock copolymers such as styrene-isoprene-styrene (SIS), styrene-butadiene-styrene (SBS), styrene-isoprene-butadiene-styrene (SIBS), styrene-ethylene/butylene-styrene (SEBS), or styrene-ethylene/propylene-styrene (SEPS). For example, the foam formulation can utilize KRATON, a thermoplastic elastomer available from Kraton Polymers, having a place of business located in Houston, Texas, U.S.A. In another example, the foam formulation can utilize VECTOR SIS and SBS thermoplastic elastomer available from Dexco, a division of ExxonMobil Chemical Company, having a place of business located in Houston, Texas, U.S.A. In still another example, the foam formulation can utilize SEPTON SEBS thermoplastic elastomer available from Kuraray America, Inc., having a place of business located in New York City, New York, U.S.A.

Additional suitable thermoplastic elastomers can include blends of thermoplastic elastomers with dynamic vulcanized elastomer-thermoplastic blends, thermoplastic polyether ester elastomers, ionomeric thermoplastic elastomers, thermoplastic elastic polyurethanes such as LYCRA polyurethane available from E. I. Du Pont de Nemours, having a place of business located in Wilmington, Delaware, U.S.A., and ESTANE available from Noveon, Inc., having a place of business located in Cleveland, Ohio, U.S.A., thermoplastic elastic polyamides, including polyether block amides such as PEBAX polyether block amide available from Atofina Chemicals, Inc., having a place of business located in Philadelphia, Pennsylvania, U.S.A., thermoplastic elastic polyesters such as HYTREL available from E. I. Du Pont de Nemours Company, and ARNITEL available from DSM Engineering Plastics, having a place of business located in Evansville, Indiana, U.S.A., and single-site or metallocene-catalyzed polyolefins having a density of less than about 0.89 grams/cubic centimeter such as AFFINITY metallocene polyethylene resins available from Dow Chemical Company, and combinations thereof.

As used herein, a tri-block copolymer has an ABA structure where the A represents several repeat units of type A, and B represents several repeat units of type B. As mentioned above, several examples of styrenic block copolymers are SBS, SIS, SIBS, SEBS, and SEPS. In these copolymers, the A blocks are polystyrene and the B blocks are the rubbery component. Generally, these triblock copolymers have molecular weights that can vary from the low thousands to hundreds of thousands and the styrene content can range from 5% to 75% based on the weight of the triblock copolymer. A diblock copolymer is similar to the triblock but is of an AB structure. Suitable diblocks include styrene-isoprene diblocks, which have a molecular weight of approximately one-half of the triblock molecular weight and having the same ratio of A blocks to B blocks. Diblocks with a different ratio of A to B blocks or a molecular weight larger or greater than one-half of triblock copolymers may be suitable for improving the foam formulation for producing low-density, soft, flexible, and absorbent foam utilizing polymer extrusion.

It may be particularly beneficial to include a thermoplastic elastomer having a high diblock content and high molecular weight as part of the foam formulation to extrude a low-density, soft, flexible, resilient, and absorbent thermoplastic foam. For example, the thermoplastic elastomer may have a diblock content between about 50 wt % and about 80 wt % of the total thermoplastic elastomer weight.

KRATON thermoplastic elastomers can function as a discontinuous phase in styrenic-based foams and further function as cell-opener generators when used in small amounts. However, in larger amounts, the cell-opener effect may be somewhat secondary compared to the resiliency, flexibility, elasticity, absorbency and softness imparted.

The foam formulation can also include blowing agents to aid in the foaming process and to help form a foamable melt. Blowing agents are compounds that decompose at extrusion temperatures to release large volumes of gas, volatile liquids such as refrigerants and hydrocarbons, ambient gases such as nitrogen and carbon dioxide, water, or combinations thereof. Both physical and chemical blowing agents, including both inorganic and organic physical blowing agents, can be used to create or enhance foaming.

Suitable inorganic physical blowing agents include water, nitrogen, carbon dioxide, air, argon, and helium. Suitable organic blowing agents include hydrocarbons such as methane, ethane, propane, butanes, pentanes, hexanes, and the like. Aliphatic alcohols and halogenated hydrocarbons including various Freon and fluorocarbons such R-134A can also be used (although their use may be avoided for environmental reasons). Endothermic and exothermic chemical blowing agents which are typically added at the extruder hopper include azodicarbonamide, paratoluene sulfonyl hydrazide, azodiisobutyro-nitrile, benzene sulfonyl hydrazide, P-toluene sulfonyl hydrazide, barium azodicarboxylate, sodium bicarbonate, sodium carbonate, ammonium carbonate, citric acid, toluene sulfonyl semicarbazide, dinitroso-pentamethylene-tetramine, phenyltetrazole sodium borohydride, and the like.

In addition, mixtures and combinations of various physical and chemical blowing agents can be used to control cell structure. Blowing agent activators can also be added to lower the decomposition temperature/profile of such chemical blowing agents. Such blowing agent activators include metals in the form of salts, oxides, or organometallic complexes.

Blowing agents can be added directly to the foam formulation or, alternatively, can be added after the melt has been heated to a temperature at or above its glass transition temperature or melting temperature. The inlet for a blowing agent, such as in an extrusion process (not shown), is typically between the metering and mixing zones. The blowing agent is then mixed thoroughly with the melted polymer at a sufficiently elevated pressure to prevent melt expansion. For example, a blowing agent can be added to the foam formulation in an amount between about 1 wt % and about 10 wt %.

Other additives can also be included in the foam formulation to enhance various properties. For example, a nucleating agent, or nucleant, can be utilized to improve foam gas bubble formation and to obtain desired fine open-cell structure. Examples of suitable nucleants include talc, magnesium carbonate, nanoclay, silica, calcium carbonate, blends of citric acid and sodium bicarbonate, coated citric acid/sodium bicarbonate particles, silica, barium stearate, diatomaceous earth, titanium dioxide, pulverized wood, clay, calcium stearate, stearic acid, salicylic acid, fatty acids, metal oxides, modified nucleant complexes, and combinations thereof. An example of a commercially available nucleant is a nanoclay available under the trade name CLOISITE® 20A, available from Southern Clay Products, Inc., having a place of business located in Gonzales, Texas, U.S.A. Various thermoplastic polymers may also be used for such purposes.

Nucleants are typically dry blended or added with the polymer concentrate. The amount of nucleant will vary based upon several parameters, including the cell structure desired, foaming temperature, pressure, polymer composition, and type of nucleating agent utilized. For example, a nucleant can be added to the foam formulation in an amount between about 0.1 wt % and about 5 wt %. Typically, as the amount of nucleant increases, the cell density likewise increases.

Still other additives that can be utilized include surface active agents (i.e., surfactants). Surfactants may be utilized to control properties such as surface tension, foam formation, and wettability.

In general, while forming the foam composite, the bubble walls may tend to drain due to factors such as gravity and capillary forces. Such drainage often thins the walls before the cell struts, or ribs, are sufficiently hardened, which in turn can result in cell collapse. La Place and Young proposed that capillary pressure at the junction of two or more struts tends to be lower, thereby creating flow from the membrane to the struts and, consequently, thinning the cell membrane. With a sufficient amount of surfactant molecules arranged preferentially to migrate to the surface of the film membrane, the presence of surfactant at the membrane's thin film surfaces may provide resistance to drainage of the molten plastic. If the film layer is sufficiently thick, such as in a foam cell membrane, it can be further stabilized by an ionic double layer of molecules resulting from orientation of ionic surfactants. Both nonionic and ionic surfactants can exhibit another stabilizing force if the membrane is sufficiently thin. This can be accomplished through alignment of surfactant tails to create a bi-layer structure, such as that found in biological cells, which are held together by Van der Waals forces, thus stabilizing the foam cell membrane. Further discussion can be found in Polymeric Foams, edited by Daniel Klempner and Kurt Frisch, Hanser Publishers, 1991, Foam Extrusion, edited by S. T. Lee, Technomic Publishing Co., Inc., 2000, Polymeric Foams, edited by S. T. Lee and N. S. Ramesh, CRC Press, 2004, and Polymeric Foams and Foam Technology, 2nd Edition, edited by Daniel Klempner and Vahid Sendijarevic, 2004, each of which is incorporated herein by reference in a manner that is consistent herewith.

While not intending to be limited to a particular theory, it is believed that a surfactant also provides resistance to diffusion of gas from a foam cell to the surroundings, which aids in resisting collapse. The reduced gas permeability due to the drainage resistance is related to the degree that a surfactant can pack into a foam cell's film surface and might explain the difference between the performances of the various surfactants. This reduced rate of diffusion allows sufficient cooling for strut formation to prevent coalescence. The surfactant does not necessarily need to completely prevent drainage, but rather can slow it sufficiently so that cell struts are substantially cooled and hardened, thereby preventing cell coalescence. In general, surfactants which tend to be highly mobile in the melt, highly surface active, and/or can pack tightly to help prevent membrane drainage will typically provide superior cell stabilization.

Suitable surfactants for the absorbent composite can be single-component or multi-component surfactants. A multi-component surfactant is a combination of two or more surfactants. It has been found that certain multi-component surfactants can achieve equal or better foam formation at a lower dosage than certain single-component surfactants. For example, in some aspects, foams utilizing a multi-component surfactant have densities comparable to foams made with over three times the amount of a single-component surfactant. Since surfactant tends to be a costly additive, the use of certain multi-component surfactants can result in foam composites having comparable foam properties at a lower cost than foams which include higher amounts of single-component surfactant.

Surfactants can be added at various locations in the foam-making process, such as directly in the foam formulation, in the composition during the foaming process, and/or as a post-treatment after formation of the foam composite. For example, a surfactant can be added to the foam formulation in a gaseous phase, such as through the use of a blowing agent (e.g., supercritical carbon dioxide).

Examples of suitable surfactants include cationic, anionic (including alkylsulfonates), amphoteric, and nonionic surfactants. Exemplary surfactants include SCHERCOPOL™ OMS-NA, a disodium momooleamido MEA sulfosuccinate, available from Scher Chemicals, Inc., having a place of business located in Clifton, New Jersey, U.S.A., and PLURONIC F68, a polypropylene glycol non-ionic surfactant which is a block copolymer of propylene oxide and ethylene oxide, available from BASF Corporation. Other examples include HOSTASTAT HS-1, available from Clariant Corporation, having a place of business located in Winchester, Virginia, U.S.A., EMEREST 2650, EMEREST 2648, and EMEREST 3712, each available from Cognis Corporation, having a place of business located in Cincinnati, Ohio, U.S.A., and DOW CORNING 193, available from Dow Corning Corporation, having a place of business located in Midland, Michigan, U.S.A. Alkyl sulfonates can also be suitable as a surfactant, although use of this class of surfactants in certain applications may be limited because of product safety concerns. However, some combinations of surfactants offer benefits where an alkyl sulfonate is added at a substantially lower level in conjunction with another surfactant to yield good foaming and wettability.

The amount of surfactant utilized will vary depending upon the particular surfactant, as well as the properties desired. For example, the surfactant can be utilized in the foam formulation in an amount between about 0.05 wt % and about 10 wt %, such as between about 0.1 wt % and about 5 wt %. In one particular example, the surfactant can be a multi-component surfactant utilized in the foam formulation in an amount between about 0.05 wt % and about 8.0 wt %, such as between about 0.1 wt % and about 3.0 wt %.

Various other additives such as lubricants, acid scavengers, stabilizers, colorants, adhesive promoters, fillers, smart-chemicals, foam regulators, various UV/infrared radiation stabilizing agents, antioxidants, flame retardants, smoke suppressants, anti-shrinking agents, thermal stabilizers, rubbers (including thermosets), anti-statics, permeability modifiers, and other processing and extrusion aids including mold release agents, anti-blocking agents, and the like can also be added to the foam formulation. In addition, fibers (e.g., wood fibers) can be added to the foam formulation to enhance water (e.g., perspiration) absorption.

In one non-limiting exemplary embodiment, the foam can comprise a thermoplastic foam derived from a foam formulation comprising about 50 wt % to about 95 wt % alkenyl aromatic base resin, about 10 wt % to about 50 wt % thermoplastic elastomer which has a styrenic block copolymer content of about 50 wt % to about 80 wt % of the elastomer, about 0.05 wt % to about 10 wt % surfactant, and about 0 wt % and about 10 wt % blowing agent.

Once the desired ingredients of the foam formulation have been determined, the materials can be added together and prepared to be formed in a foam-making process, including those foam-making processes known by persons having ordinary skill in the art. For example, various continuous plastic extrusion processes known in the art can be utilized to produce the foam. Other suitable foam-making processes known in the art include injection molding, batch processes, and air-forming processes.

In general, the materials can be heated such that the materials form a molten foam melt, at which time the materials can form a substantially homogeneous mixture. In some aspects, the materials are suitably heated to a temperature between about 100° C. and about 500° C. to create the foam melt. Such foam melt can then be foamed to create cells within the melt using suitable foaming techniques known to persons having ordinary skill in the art. Once formed, the foam melt can then be processed, such as with an extrusion process, and cooled to form a foam mitigation member 310.

In some aspects, continuous plastic extrusion processes known in the art can be utilized to produce a foam mitigation member 310. In the case of such extrusion processes, a tandem screw-type extruder can be utilized. This type of extruder may be considered particularly suitable in some aspects because it has the ability to provide tight control of extrusion temperatures to produce open-cell foams. With tandem extruders, the first extruder section typically contains several zones including: feed and conveying, compression, melting, metering and mixing zones and the second extruder section often contains a cooling zone and a shaping zone prior to the discharge. The first extruder is typically hopper loaded with the base resin(s) and thermoplastic elastomer(s), as well as any other optional additives. Techniques known in the art for accomplishing this include using dry/blend/metering equipment and/or having the components incorporated into a pelletized polymer concentrate such as in a master batch. The components of the foam formulation are then heated in the extruder to form a plasticized or melt polymer system, often with zoned temperature control using an extruder's cooling/heating systems.

The foamable melt is then typically cooled to a lower temperature to control the desired foam cell structure. In the case of tandem extruders, the cooling is typically accomplished in the second extruder which is connected downstream of the first extruder through a heated cross-over supply pipe. In the case of single extruders, cooling is typically accomplished upstream of the discharge orifice. Often cooling/heating systems with process temperature control loops are incorporated to tightly control foam bubble nucleation/growth within the gas-laden melt. The optimum cooling temperature is typically at or slightly above the glass transition temperature or melting point of the melt.

The melt is then extruded through a die to a lower pressure (typically atmospheric or a vacuum) to cause thermodynamic instability and foaming which then cools and crystallizes the plastic to form a stabilized foam which then solidifies to form a web or layer. Often circular, annular or slit dies, including curtain dies, and the like are used, often with a mandrel, to shape and draw the web to the desired gauge, shape and orientation with foam expansion and cooling.

Various equipment configurations using such extrusion can be used to manufacture a foam mitigation member 310 of the present invention. In addition, various specialized equipment can be employed upstream of specially designed dies to enhance mixing, cooling, cellular structure, metering, and foaming. Such equipment includes static mixers, gear pumps, and various extruder screw designs, for example. Stretching equipment, including roller nips, tenters, and belts, may also be used immediately downstream of the discharge to elongate cellular shape to enhance absorbency. Microwave irradiation for cross-linking, foaming activation, and mechanical means can also be used to enhance foam properties. Foam contouring, shaping (e.g. patterning, perforating, etc.) and the like, using thermoforming, and other such thermal processes, including thermal bonding, can be used to control shaping, flexibility, softness, aesthetics, and absorbent swelling.

Open-cell formation can be regulated by elevated processing pressures and/or temperatures, as well as by using additives such as nucleating agents, chemical blowing agents, and low additions of immiscible polymers, and/or surfactants which can control both cell density and cell structure. Particular base resins are also sometimes used to broaden the foaming temperature to make open-cell foam. For example, the open-cell level of a polystyrenic-based foam can be facilitated by adding small amounts of various immiscible polymers to the foam formulation, such as by adding polyethylene or ethylene/vinyl acetate copolymer, to create interphase domains that cause cell wall rupture. In another example, ethylene-styrene interpolymers can be added to alkenyl aromatic polymers to control open-cell quality and improve surface quality and processability. In still another example, small amounts of polystyrene-based polymers can be added to polyolefin-based foams to increase open-cell content. The open-cell content and microporous cell membrane uniformity can also be controlled by regulating the polymer system components and crystallization initiating temperature.

Suitable foams may be available commercially. For example, foams which retain bulk thickness after hydraulic needling (i.e., resilient foams) include RYNEL 562-B medical grade polyurethane and RYNEL 562-D medical grade polyurethane, both available from Rynel Ltd., Inc., a division of Mölnlycke Health Care AB, having a place of business located in Gothenburg, Sweden. Other suitable foam layers include MINICELL STD crossed-linked polyethylene, available from Voltek, a division of Sekisui America Corporation, having a place of business located in Lawrence, Massachusetts, U.S.A., latex foams such as those described in U.S. Pat. No. 6,627,670 to Mork et al., which is incorporated herein by reference in a manner that is consistent herewith, High Internal Phase Emulsion (HIPE) foams such as those described in U.S. Pat. No. 5,260,345 to DesMarais et al., which is incorporated herein by reference in a manner that is consistent herewith, and extruded thermoplastic foams such as those described in U.S. Pat. No. 7,358,282 to Krueger et al. and U.S. Pat. No. 6,071,580 to Bland et al., each of which is incorporated herein by reference in a manner that is consistent herewith.

In addition to the above, secondary post-treatment processes can be performed to provide or enhance desirable properties including, inter alia, perforating, softening, flexibility, absorbency, cellular orientation, aesthetics, and the like. This can be accomplished through numerous techniques known in the art including mechanical needling and other mechanical perforation, stretching and drawing, calendaring or creping, brushing, scarfing, buffing/sanding, and thermoforming/shaping. Often a foam surface skin may form during extrusion, which can later be skived or sliced off, needle-punched, brushed, scraped, buffed, scarved, sanded, or perforated to remove the barrier, or portions thereof. Mechanical, hydraulic, thermal, or laser perforation can also be utilized. Mechanical, laser, and/or hydraulic micro-serrations can also be employed (e.g., to enhance permeability). In addition, application of a surfactant after the foaming process or needling process may further be utilized to afford a desired wettability.

Processes can be utilized for making open-cell foams, low-density foams, absorbent foams, and soft, resilient, elastomeric foams. Some examples of such processes are described in U.S. Pat. No. 5,962,545 to Chaudhary et al., U.S. Pat. No. 5,728,406 to Halberstadt et al., and U.S. Pat. No. 6,451,865 to Migchels et al., each of which is incorporated herein by reference in a manner that is consistent herewith.

Plasticizing agents are sometimes used as cell openers in producing foams. When used as cell openers, such plasticizing agents are added to the foam formulation in minor amounts, such as described in U.S. Pat. No. 6,071,580 to Bland et al., which is incorporated herein by reference in a manner that is consistent herewith. More particularly, the plasticizing agent can act to increase cell expansion to produce a high expansion ratio. When cells expand, membranes between cells thin and can become unstable, rupture, and can thereby create porous connections between cells. In addition, when thermoplastic polymer cools and with volumetric contraction with crystallization, thin portions of the membrane can rupture enough to create additional connections or pores between cells, thereby creating open cells.

Although plasticizing agents act as softeners, the addition of plasticizing agents makes foaming to low densities more difficult. For example, in a high density, essentially closed-cell, non-absorbing foam containing a plasticizing agent and thermoplastic elastomer and an additive such as a surfactant, plasticizing agents can lower polymer melt viscosities and lead to increasing melt drainage which causes foaming difficulties with cell collapse, such as described in U.S. Pat. No. 6,653,360 to Gupta, which is incorporated herein by reference in a manner that is consistent herewith.

There is a wide range of plasticizing agents available. The desired properties for selecting a plasticizing agent includes not only its softening ability, but also temperature stability upon extrusion, resistance to migration, cost, odor, biodegradability, and manufacturing and consumer safety. Typical plasticizing agents include citrates, phthalates, stearates, fats and oils. It is known that glycerol fatty acids, such as glycerol monostearate, stabilize cells by reducing the rate of gas diffusion from the cell.

Accordingly, in some aspects, a plasticizing agent can be included in the foam formulation. A plasticizing agent is a chemical agent that imparts flexibility, stretchability and workability. The type of plasticizing agent has an influence on foam gel properties, blowing agent migration resistance, cellular structure, including fine cell size and number of open cells. Typically, desirable plasticizing agents are of low molecular weight (e.g., less than 1,000). The increase in polymer chain mobility and free volume caused by incorporation of a plasticizing agent typically results in a Tg decrease, and plasticizing agent effectiveness is often characterized by this measurement. Petroleum-based oils, fatty acids, and esters are commonly used and act as external plasticizing agents or solvents because they do not chemically bond to the polymer, yet remain intact in the polymer matrix upon crystallization.

The plasticizing agent increases cell connectivity by thinning membranes between cells to the point of creating porous connections between cells, thus the plasticizing agent increases open-cell content. If desired, a plasticizing agent can be included in the foam formulation in an amount of about 0.5 wt % to about 10 wt %, such as about 1 wt % and about 10 wt %. Such plasticizing agent should be gradually and carefully metered in increasing concentration into the foam formulation during the foaming process as too much plasticizing agent added at once can create cellular instability, resulting in cellular collapse.

Other examples of suitable plasticizing agents can include polyethylene, ethylene vinyl acetate, mineral oil, palm oil, waxes, esters based on alcohols and organic acids, naphthalene oil, paraffin oil, and combinations thereof. A commercially available plasticizing agent is a small-chain polyethylene that is produced as a catalytic polymerization of ethylene, which is often referred to in the art as a “wax” because of its low molecular weight. An example of such low-density, highly branched polyethylene “wax” is EPOLENE C-10 available from Eastman Chemical Company, having a place of business located in Kingsport, Tennessee, U.S.A.

Still other examples of plasticizing agents include acetyl tributyl citrate, acetyl triethyl citrate, p-tert-butylphenyl salicylate, butyl stearate, butylphthalyl butyl glycolate, dibutyl sebacate, di-(2-ethylhexyl) phthalate, diethyl phthalate, diisobutyl adipate, diisooctyl phthalate, diphenyl-2-ethylhexyl phosphate, epoxidized soybean oil, ethylphthalyl ethyl glycolate, glycerol monooleate, monoisopropyl citrate, mono-, di-, and tristearyl citrate, triacetin (glycerol triacetate), triethyl citrate, and 3-(2-xenoyl)-1,2-epoxypropane.

With continuing reference to FIGS. 5A-5B, as referenced above, the mitigation member 310 can optionally comprise one or more apertures 320. The purpose of such optional apertures 320 is to provide tunnels or channels 322 into or through the mitigation member 310 (typically in the general direction of the z-axis 3) which allow the reaction media to migrate towards the bottom side 312 of the mitigation member 310. In such embodiments, at least a portion of the thermal-conductive polymer 330 can extend through the entire thickness (as measured along the z-axis 3) of the mitigation member 310. Such channels 322 can have any functional cross-sectional shape profile as would be known to persons having ordinary skill in the art, such as circular, oval, square, rectangular, star-shaped, random, and the like, and combinations thereof. The apertures 320 can comprise any suitable dimensions. For example, in the case of apertures 320 having a circular shape-profile, the resulting cylindrical channels 322 can comprise a cross-sectional diameter of about 1 mm to about 10 mm, such as about 2 mm to about 7 mm, or about 3 mm to about 4 mm to provide improved benefits. However, it should be understood that the dimensions can be less than 1 mm or greater than 10 mm without departing from the scope of the invention.

With further reference to FIGS. 4A-6B, depending upon the characteristics of the particular mitigation member 310 and the characteristics of the thermal-conductive polymer 330, the cross-sectional dimensions of any channels 322 present in the mitigation member 310 will tend to increase or enlarge (i.e., the volume of the channels 322 will expand) upon entry of the reaction media from which the thermal-conductive polymer 330 is derived. Such expansion can typically range between about 50% to about 500%, such as about 100% to about 400%, or about 200% to about 400%, for example. It should be understood that such expansion may be less than 50% or greater than 500% without departing from the scope of the invention. In addition, it should be understood that the expansion of each channel 322 created by the apertures 320 need not be equivalent, but rather may vary throughout the mitigation member 310 without departing from the scope of the invention. In general, a comparatively greater increase in a particular channel 322 dimension as compared to another channel will indicate a greater amount by weight and/or volume of polymer 330 present in that channel 322.

Continuing with FIGS. 4A-6B, as referenced above, the therapeutic member 300 also comprises a thermal-conductive polymer 330. The purpose of the thermal-conductive polymer 330 includes, inter alia, conducting thermal energy (e.g., heat) away from the user, providing cushioning support to the user, attenuating forceful impacts, and reducing or eliminating pressure points. The thermal-conductive polymer 330 may be present upon the top side 311 of the mitigation member 310 and/or can be disposed at least partially through the thickness of the mitigation member 310 (i.e., migrating from the top side 311 towards the bottom side 312). The amount of thermal-conductive polymer 330 present upon the top side 311 of the mitigation member 310 will depend upon, inter alia, the overall quantity of reaction media applied to the mitigation member 310, the quantity of liquid reaction media that migrates into and/or through the thickness of the mitigation member 310, the absorbency (e.g., open cell content) of the mitigation member 310, the viscosity of the reaction media, the curing rate of the reaction media, the size of any optional apertures 320 which may be disposed within the mitigation member 310, etc.

While still in liquid form (i.e., uncured or partially cured), the reaction media can be relatively evenly applied to the top side 311 of a suitable mitigation member 310 (e.g., a soft, resilient, elastomeric foam substrate comprising a plurality of optional apertures 320), and then allowed to fully cure, thus forming one exemplary embodiment of an inventive therapeutic member 300 of the present disclosure. Suitable methods for applying the reaction media to the mitigation member 310 include those known to persons having ordinary skill in the art, such as pouring, spraying, printing, injecting, and the like.

In some aspects, the reaction media will completely soak into the mitigation member 310. In other aspects, the reaction media may cure to form an optional polymer layer 335 upon the top side 311 of the mitigation member 310. Typically, the thickness (as measured along the z-axis 3) of such polymer layer 335 will range from about 0 mm to about 5 mm. However, it should be understood that the thickness of a polymer layer 335 which forms upon the top side 311 of the mitigation member 310 can be greater than 5 mm without departing from the scope of the invention.

The amount of thermal-conductive polymer 330 utilized with the inventive therapeutic member 300 will vary depending upon the desired properties. Typically, the amount of thermal-conductive polymer 330 will range from about 1 gram polymer per gram mitigation member 310 (1 g/g) to about 10 grams polymer per gram mitigation member 310 (10 g/g). However, it should be understood that less than 1 g/g or greater than 10 g/g can also be suitable without departing from the scope of the invention.

Some examples of suitable polymers for forming the thermal-conductive polymer 330 of the present invention are described in U.S. Pat. No. 7,041,719 to Kriesel et al., U.S. Pat. No. 11,124,596 to Kriesel et al., U.S. patent application Ser. No. 14/756,152 to Goodenough, and U.S. patent application Ser. No. 17/460,196 to Kriesel et al., each of which is incorporated herein by reference in a manner that is consistent herewith. Variations of such polymer, as well as other polymers having similar properties, including silicone-based polymers, can also be suitable for the present invention without departing from the scope of the invention.

By way of a non-limiting example, a reaction media to produce the thermal-conductive polymer 330 can be prepared comprising about 2 percent by weight of the total reaction media weight (wt %) to about 20 wt % prepolymer (e.g., isocyanate prepolymer, silicone prepolymer, etc.), about 20 wt % to about 40 wt % hydroxyl functional thermoplastic elastomer, and greater than about 40 wt % epoxidized triglyceride plasticizer. In another non-limiting example, a reaction media can be prepared comprising about 2 percent by weight of the total reaction media weight (wt %) to about 20 wt % prepolymer, about 1 wt % to about 65 wt % straight chain polyols, about 3 wt % to about 50 wt % crosslinking polyols, about 40 wt % to about 80 wt % epoxidized triglyceride plasticizer, and 0 wt % to about 40 wt % viscosity reducing plasticizer. In some aspects, the prepolymer can comprise an isocyanate prepolymer (e.g., diisocyanate), a silicone prepolymer, or the like. In some aspects, the straight chain polyols can comprise a diol (e.g., polyether diol). In some aspects, the crosslinking polyols can comprise a triol or higher (e.g., polyether triol). In some aspects, the epoxidized triglyceride plasticizer can comprise an epoxidized vegetable oil plasticizer (e.g., epoxidized soybean oil plasticizer). In some aspects, the optional viscosity reducing plasticizer can comprise an ester plasticizer. In some aspects, the reaction media can be reacted in the presence of about 0.001 wt % to about 5 wt % catalyst (e.g., a tin based catalyst).

As referenced above, in some aspects the reaction media which forms the thermal-conductive polymer 330 can comprise a quantity of prepolymer which forms the backbone of the polymer 330. Such prepolymer will typically be present in an amount of about 2 wt % to about 20 wt % of the total reaction media weight. Suitable prepolymers can include a ring-opening species of a hardener (e.g., amines, amides, mercaptans, anhydrides, isocyanates including polyisocyanates (such as a diisocyanate), etc.). Suitable polyisocyanates include, but are not limited to, aromatic diisocyanates (e.g., diphenylmethane diisocyanate, methylene diphenyl diisocyanate (MDI), toluene diisocyanate (TDI), etc.) and aliphatic diisocyanates (e.g., hexamethylene diisocyanate (HDI), isophorone diisocyanate (IPDI), etc.) in a conventional prepolymer form. In one non-limiting example, a methylene diphenyl diisocyanate (MDI) designated as ELASTOCAST TQZ-P23, available from BASF Corporation, can provide a suitable prepolymer to form the thermal-conductive polymer 330 of the present disclosure.

As referenced above, in some aspects, the reaction media which forms the thermal-conductive polymer 330 can comprise a thermoplastic elastomer. Suitable thermoplastic elastomers can include most any thermoplastic compound having elastomeric properties. In some preferred aspects, such resins or thermoplastics can have primary allylic alcohol groups that exhibit high reactivity in condensation polymerization reactions. Some suitable thermoplastic elastomers can include, but are not limited to, polydienes (e.g., polybutadiene). In one non-limiting example, a suitable thermoplastic elastomer includes POLY BD R45 HTLO and POLY BD R45 V, each of which is a low molecular weight hydroxyl terminated polybutadiene resin available from Cray Valley.

As referenced above, in some aspects, the reaction media which forms the thermal-conductive polymer 330 can comprise a quantity of polyols, typically ranging from about 10 wt % to about 75 wt % of the total reaction media weight. More particularly, such polyols can include straight chain polyols and crosslinking polyols. In some desirable aspects, the straight chain polyols can be in the form of diols (e.g., a polyol having two terminal reactive groups), and the crosslinking polyols can be in the form of triols or higher (e.g., a polyol having two terminal reactive groups and at least one additional reactive group). Such straight chain polyols and crosslinking polyols are preferably liquids at room temperature (i.e., about 21° C.) and generally have a molecular weight of about 1,000 to about 20,000. The cohesiveness of the resulting thermal-conductive polymer 330 depends upon using a controlled polyol balance (i.e., straight chain polyols and crosslinking polyols) within the reaction media. It has been discovered herein that the amount of straight chain polyols and crosslinking polyols (preferably reacted in the presence of an effective amount of plasticizer within the reaction media) can suitably fall within a prescribed straight chain polyol to crosslinking polyol weight ratio of about 1:3 to about 3:1, such as about 1:2 to 2:1, or about 7:13 to about 13:7, to provide desired viscoelastic and cohesive attributes. In general, decreasing the straight chain polyol to crosslinking polyol ratio (i.e., increasing the crosslinking polyol content relative to the straight chain polyol content) will generally result in an increased cohesiveness of the thermal-conductive polymer 330.

In general, a diol can provide straight chain infrastructure formation and sufficient crosslinkage disruption to permit for a highly effective intermolecular plasticizer attraction and alignment, thus providing for an unusually high and effective loading of plasticizer. In some preferred aspects, the straight chain diol can be provided by a polyether diol having a molecular weight ranging from about 1,000 to about 10,000, such as about 1,000 to about 8,000, or about 2,000 to about 6,000 for improved benefits, and preferably having two (2) terminal reactive groups (e.g., hydroxyl groups). The straight chain polyol component of the reaction media can be suitably present in an amount ranging from about 1 wt % to about 65 wt % of the total reaction media weight, such as about 3 wt % to about 35 wt %, or about 5 wt % to about 15 wt % of the total reaction media weight, to provide improved benefits. In one example, a 2-functional polyether diol, designated as ELASTOCAST C-4057, available from BASF Corporation, can provide a suitable straight chain polyol component to form the thermal-conductive polymer 330 component of the therapeutic member 300.

In general, a crosslinking polyol (e.g., triol) can provide sufficient crosslinkage infrastructure to the polymer 330, and can contribute to the cohesiveness thereof. In some preferred aspects, the crosslinking polyol can be provided by a polyether triol having a molecular weight ranging from about 1,000 to about 10,000, and preferably having three (3) reactive groups (e.g., hydroxyl groups) wherein two (2) of the reactive groups are terminal reactive groups. The crosslinking polyol component of the reaction media can be suitably present in an amount ranging from about 3 wt % to about 50 wt % of the total reaction media weight, such as about 10 wt % to about 45 wt %, or about 20 wt % to about 40 wt % of the total reaction media weight, to provide improved benefits. In one example, a 3-functional polyether triol, designated as ELASTOCAST C-4018, available from BASF Corporation, can provide a suitable crosslinking polyol component to form the thermal-conductive polymer 330 component of the therapeutic member 300.

As referenced above, in some aspects, the reaction media which forms the thermal-conductive polymer 330 can comprise a quantity of plasticizer. Typically, the amount of total plasticizer will be greater than about 40 wt % of the total reaction media weight, such as about 40 wt % to about 80 wt % or about 45 wt % to about 70 wt % to provide improved benefits. More particularly, the plasticizer can include an epoxidized triglyceride plasticizer, and can optionally further include a viscosity reducing plasticizer, preferably an ester plasticizer. The plasticizer components are preferably liquids at room temperature (i.e., about 21° C.). A controlled amount of epoxidized triglyceride plasticizer and optional viscosity reducing plasticizer (e.g., ester plasticizer) within the prescribed range can provide an effective reaction media for preparing a thermal-conductive polymer 330 possessing the desired compositional attributes for use herein. Desirably, the plasticizer component is uniformly dispersed and cohesively bound throughout the reaction media (along with the other polymerizable components) and will tenaciously remain uniformly dispersed within the resultant thermal-conductive polymer 330 in a cohesive and stabilized form.

Suitable epoxidized triglyceride plasticizers include epoxidized animal oils and epoxidized vegetable oils. Amongst the suitable epoxidized triglyceride plasticizers, epoxidized vegetable oils (e.g., epoxidized soybean oil, epoxidized castor oil, epoxidized corn oil, epoxidized cottonseed oil, epoxidized perilla oil, epoxidized safflower oil, epoxidized linseed oil, epoxidized tall oil, etc.) are particularly effective epoxidized triglyceride plasticizers. Other suitable epoxidized triglyceride plasticizers have been more extensively described in the aforementioned patents and applications which have been incorporated herein by reference. Such epoxidized triglyceride plasticizers can be suitably present in an amount that is greater than 40 wt % of the total reaction media weight, such as about 40 wt % to about 80 wt %, or about 45 wt % to about 70 wt % of the total reaction media weight. In one desirable example, epoxidized soybean oil can provide a highly suitable epoxidized triglyceride plasticizer to form the thermal-conductive polymer 330 component of the therapeutic member 300.

As referenced above, in some aspects, the reaction media which forms the thermal-conductive polymer 330 can also optionally comprise a suitable reaction media viscosity reducing plasticizer. In general, plasticizers which are suitable as plasticizing agents for the plasticization of polyvinyl chlorides can be utilized as viscosity reducing plasticizers for the reaction media. Typically, an optional viscosity reducing plasticizer will be present in an amount of about 0 wt % to about 40 wt % of the total reaction media weight.

Exemplary viscosity reducing plasticizers for preparing the thermal-conductive polymer 330 can include, but are not limited to, ester plasticizers. Such ester plasticizers are especially effective as an optional plasticizer component in the reaction media. Suitable ester plasticizers have a relatively low molecular weight, typically less than about 750, such as less than about 500, and can include, but are not limited to, the condensation products of alcohols (e.g., C₁-C₁₀ alcohols, such as C₂-C₆ alcohols) and dicarboxylic acids (e.g., C₂-C₁₂ dicarboxylic acids, such as C₄-C₈ dicarboxylic acids). In addition, amongst the more fluid ester plasticizers, such as diester plasticizers for example, are the lower dialkyl esters of dicarboxylic acids, such as dialkyl esters having alkyl groupings of less than 12 carbon atoms, such as C₁-C₈ dialkyl ester groupings of sebacates, adipates, phthalates, isophthalates, maleates, azelates, glutarates, etc.

In some aspects, the incorporation of the optional relatively low molecular weight ester plasticizer in combination with the epoxidized triglyceride plasticizer can be utilized to provide an easier fabricating form (e.g., for casting, molding, injecting, pouring, etc.) of the reaction media by lowering the viscosity of the reaction media without adversely affecting the desirable features of the thermal-conductive polymer 330. For example, the addition of polar ester plasticizers, or substitution of the epoxidized triglyceride plasticizers with polar ester plasticizers, has been found to effectively reduce the viscosity of the reaction media while still maintaining a desired level of heat dissipation, cohesiveness, viscoelasticity, and impact dispersion of the resulting polymer 330, as well as excellent stability properties. It has been discovered herein that including an ester plasticizer having a fluid (i.e., liquid) consistency at room temperature (i.e., about 21° C.) and having a relatively low molecular weight (e.g., less than about 750) in the reaction media can contribute to ideal working viscosities during the initial curing stages, rendering the reaction media to be more effective for forming the thermal-conductive polymer 330 component of the therapeutic member 300.

In aspects comprising a viscosity reducing plasticizer, the total quantity of all plasticizers can be about 40 wt % or greater of the total reaction media weight, such as about 40 wt % to about 85 wt % of the total reaction media weight, such as at least 45 wt % to about 75 wt %, or about 50 wt % to about 70 wt %, to provide improved benefits. The plasticizer components of the reaction media are typically liquids at room temperature (i.e., about 21° C.). In such embodiments, it has been discovered herein that a weight ratio of epoxidized triglyceride plasticizer to viscosity reducing plasticizer can suitably fall within a range of about 1:0 to about 1:1, such as about 6:1 to about 1:3, or about 3:1 to about 1:2, to provide a workable reaction media viscosity, and to help provide the desired cohesiveness, viscoelastic, impact dispersing and heat dissipating attributes of the resulting polymer 330.

It should be understood that the reaction media which forms the thermal-conductive polymer 330 can also optionally comprise additional constituents including, but not limited to, catalysts, initiators, other additional plasticizers, colorants, UV inhibitors, antioxidants, and the like, as would be known to persons having ordinary skill in the art, without departing from the scope of the invention. For example, the polymerization of the reaction media can be carried out in the presence of a catalyzing amount (defined above) of a catalyst (preferably a slow-acting catalyst or a heat-activated catalyst) to control the curing rate of the reaction media. Such catalyst is typically employed in relatively small amounts, such as about 0.001 wt % to about 5 wt % of the total reaction media weight.

Suitable catalysts can include tertiary amines, tertiary phosphines, strong bases (e.g., alkali, alkaline earth metal hydroxides, alkoxides, phenoxides, etc.), acidic metal salts of strong acids, metal chelates, metal alcoholates, metal phenolates, organic acid salts, organo metallic derivatives, etc. In one non-limiting example, a slow-acting organobismuth catalyst, available under the trade name COSCAT 83 (available from Vertellus Holdings LLC, having a place of business located in Zeeland, Michigan, U.S.A.), can provide a suitable catalyst for controlling the curing rate of the thermosetting reaction media to form the thermal-conductive polymer 330 component of the therapeutic member 300. In another non-limiting example, a heat-activated tin thioglycolate catalyst, available under the trade names FOMREZ CATALYST UL-29 and FOMREZ CATALYST UL-54 (each available from Momentive Performance Materials Inc., having a place of business located in Wilton, Connecticut, U.S.A.), can provide a suitable catalyst for controlling the curing rate of the thermosetting reaction media to form the thermal-conductive polymer 330 component of the therapeutic member 300.

Other suitable catalysts can include: (a) tertiary amines such as bis(dimethylaminoethyl) ether, trimethylamine, triethylamine, N-methylmorpholine, N-ethylmorpholine, N,N-dimethylbenzylamine, N,N-dimethylethanolamine, N,N,N′,N′-tetramethyl-1,3-butanediamine, triethanolamine, 1,4-diazabicyclo[2.2.-2]octane, N,N-dimethylcyclohexylamine, N-methyldicyclohexylamine, 1,8-diazabicyclo[5,4,0]-undecene-7 and its salts such as phenol salt, hexanoate, and oleate, 2,4,6-tris(diaminomethyl) phenol, and the like, (b) tertiary phosphines such as trialkylphosphines, dialkylbenzylphosphines, and the like, (c) strong bases such as alkali and alkaline earth metal hydroxides, alkoxides, and phenoxides, (d) acidic metal salts of strong acids such as ferric chloride, stannic chloride, stannous chloride, antimony trichloride, bismuth nitrate and chloride, and the like, (e) chelates of various metals such as those which can be obtained from acetylacetone, benzoylacetone, trifluoroacetylacetone, ethyl acetoacetate, salicylaldehyde, cyclopentanone-2-carboxylate, acetylacetone-imine, bis-acetylacetonealkylenediimines, salicylaldehydeimine, and the like, with various metals such as Be, Mg, Zn, Cd, Pb, Ti, Zr, Sn, As, Bi, Cr, Mo, Mn, Fe, Co, Ni, or such ions as MoO++, UO++, and the like, (f) alcoholates and phenolates of various metals such as Ti(OR), Sn(OR), Al(OR), and the like, wherein R is alkyl or aryl and the reaction products of alcoholates with carboxylic acides, beta-diketones, and 2-(N,N-dialkylamino)alkanols, such as the well-known chelates of titanium, (g) salts of organic acids with a variety of metals such as alkali metals, alkaline earth meals, Al, Sn, Pb, Mn, Co, Ni, and Cu, including, for example, sodium acetate, potassium laurate, calcium hexanoate, stannous acetate, stannous octoate, stannous oleate, lead octoate, metallic driers such as manganese and cobalt naphthenate, and the like, and (h) organometallic derivates of tetravalent tin, trivalent and pentavalent As, Sb, and Bi and metal carbonyls of iron and cobalt, mercury compounds such as arylmercury carboxylates, phenylmercury acetate and propionate, and the like.

Still other suitable catalysts can include an alkyl tin compound such as dialkyltin salts of carboxylic acids, e.g., dibutyltin diacetate, dibutyltin dilaurate, dibutyltin maleate, dilauryltin diacetate, dioctyltin diacetate, dibutyl-tin-bis(4-methylaminobenzoate), dibutyltin-bis(6-methylaminocaproate), and the like. Dialkyltin mercaptides, in particular diakyltin dimercaptide carboxylic acid esters, can also be utilized. Similarly, there can be used a trialkyltin hydroxide, dialkyltin oxide, dialkyltin dialkoxide or dialkyltin dichloride. Examples of these compounds include trimethyltin hydroxide, tributyltin hydroxide, trioctyltin hydroxide, dibutyltin oxide, dioctyltin oxide, dilauryltin oxide, dibutyltin-bis(isopropoxide), dibutyltin-bis-(2-dimethylaminopentylate), dibutyltin dichloride, dioctylin dichloride, and the like. For example, a suitable alkyl tin compound is COTIN 430, a dioctyltin carboxylate available from Cambrex Company, having a place of business located in Itasca, Illinois, U.S.A.

It has been discovered herein that blend of catalysts can also be beneficial in embodiments. For instance, in one non-limiting example, a blend of COTIN 430 and FOMREZ CATALYST UL-54 was utilized with the reaction media to provide improved benefits.

Procedurally, the reaction product which forms the thermal-conductive polymer 330 can be prepared from a thermosetting reaction media homogeneously loaded with plasticizer(s) which includes an epoxidized triglyceride plasticizer, as well as optionally any other effective polar plasticizer, coupled with a carefully measured amount of straight chain polyols and crosslinking polyols (to create the necessary bridging between the crosslinks), and an isocyanate prepolymer hardener (e.g., diisocyanate, such as aliphatic, aromatic, heterocyclic, etc., polyisocyanates, cycloaliphatic isocyanates and arylaliphatic isocyanates) or silicone prepolymer, and typically in the presence of an appropriate catalyst (e.g., preferably a relatively slow acting catalyst). The reaction media desirably contains the necessary plasticizer loading specifically adapted to provide a curable reaction media, which upon curing, produces a viscoelastomeric reaction product (i.e., the thermal-conductive polymer 330) having a unique polymerizate structure effectively loaded with polar oriented plasticizers uniformly and homogeneously distributed throughout the polymer's entire thermoset mass, intertwined therewithin, and supported by the flexible plasticizer-entrapping, thermoset polymerizate structure.

With reference now to FIGS. 5A-6B, in some aspects, the therapeutic member 300 can optionally comprise a barrier layer 340. Such optional barrier layer 340 will typically be disposed upon the bottom side 312 of the mitigation member 310 (and thus the bottom side 302 of the therapeutic member 300), thus forming a laminated-type structure. The purpose of the optional barrier layer 340 includes, inter alia, halting the migration of liquid reaction media during production (i.e., substantially confining the reaction media to the channels 322 formed by optional apertures 320 versus migrating upon the entire bottom side 312 of the mitigation member 310), preventing direct contact between the thermal-conductive polymer 330 and the top side 121 of the thermal support member 120, and preventing any potential leakage of plasticizer (if any) which may occur from migrating into the thermal support member 120. Preferably, such optional barrier layer 340 will be flexible (preferably a flexibility similar to that of the mitigation member 310), resilient, elastic and/or at least partially resistant to plasticizer leakage. Suitable barrier layers 340 can desirably be in the form of a substrate, and can comprise materials known to persons having ordinary skill in the art, such as plastics (e.g., polyethylene, polypropylene, PVC, etc.), polyester, elastic webbing, Spandex, vinyl, thermoplastic foam (e.g., substantially closed-cell), thermoset foam (e.g., substantially closed-cell), natural and synthetic leather, natural and synthetic rubber, Gortex, nonwovens (e.g., meltblown, spunbond, etc.), coated woven textiles, and the like, and combinations thereof. The optional barrier layer 340 can comprise any functional thickness, but will typically be relatively thin, such as between about 0.1 mm and about 1 mm. However, it should be understood that an optional barrier layer 340 can have a thickness of less than 0.1 mm or greater than 1 mm without departing from the scope of the invention. In some preferred aspects, an optional barrier layer 340 can be adhered to the bottom side 302 of the therapeutic member 300 by virtue of being placed in contact with the reaction media while curing to form the thermal-conductive polymer 330 during production. However, an optional barrier layer 340 can be attached to the bottom side 302 of the therapeutic member 300 post-production via a suitable attachment means as would be known to persons having ordinary skill in the art (e.g., stitching, adhesives, mechanical fasteners, etc.) without departing from the scope of the invention.

Continuing with FIGS. 5A-6B, in some aspects, the therapeutic member 300 can optionally comprise a comfort layer 350. Such optional comfort layer 350 will typically be disposed upon the top side 311 of the mitigation member 310 (and thus the top side 301 of the therapeutic member 300), thus forming a laminated-type structure. The purpose of the optional comfort layer 350 includes, inter alia, adding an additional layer of comfort to the user, providing an aesthetically pleasing visual attribute, providing an aesthetically pleasing feel, buffering the user from “feeling” the presence of optional channels 322 comprising thermal-conductive polymer 330, further reducing or eliminating pressure points, preventing direct contact between the thermal-conductive polymer 330 and a bedding sheet (not shown) or an optional sheath member110 (if present), and retarding any potential leakage of plasticizer (if any) which may occur from migrating out of the inventive therapeutic member 300. Preferably, such optional comfort layer 350 will be soft (preferably a softness similar to that of the mitigation member 310), flexible (preferably a flexibility similar to that of the mitigation member 310), resilient, elastic and/or at least partially resistant to plasticizer leakage. Suitable comfort layers 350 can optionally be in the form of a substrate or pad, and can comprise materials known to persons having ordinary skill in the art, such as plastics (e.g., polyethylene, polypropylene, PVC, etc.), polyester, elastic webbing, Damask, Spandex, satin, Sateen, vinyl, thermoplastic foam, thermoset foam, Gortex, nonwovens (e.g., meltblown, spunbond, etc.), coated or uncoated woven textiles, cotton, wool, felt, natural and synthetic leather, natural and synthetic rubbers, and the like, and combinations thereof. The optional comfort layer 350 can comprise any functional thickness, but will typically be relatively thinner than the mitigation member 310, such as between about 0.1 mm and about 3 cm, or about 0.5 mm and about 1.5 cm. However, it should be understood that an optional comfort layer 350 can have a thickness of less than 0.1 mm or greater than 3 cm without departing from the scope of the invention. In some preferred aspects, an optional comfort layer 350 can be adhered to the top side 301 of the therapeutic member 300 by virtue of being placed in contact with the reaction media while curing to form the thermal-conductive polymer 330 component of the therapeutic member 300 during production. However, an optional comfort layer 350 can also be attached to the top side 301 of the therapeutic member 300 post-production via a suitable attachment means as would be known to persons having ordinary skill in the art (e.g., stitching, adhesives, mechanical fasteners, etc.) without departing from the scope of the invention.

As referenced above, in some aspects, the therapeutic member 300 can optionally comprise a sheath member 110 which can be disposed upon and/or can at least partially surround or encase the therapeutic member 300. Such flexible sheath member 110 may be desirable for numerous reasons including, inter alia, visual aesthetics, surface feel, additional structural support, plasticizer leakage barrier, keeping the various components of the inventive decubitus prevention device 100 aligned with respect to each other, etc. Suitable sheath members 110 can comprise materials known to persons having ordinary skill in the art, such as plastics (e.g., polyethylene, polypropylene, PVC, etc.), polyester, elastic webbing, Damask, Spandex, satin, Sateen, vinyl, thermoplastic foam, thermoset foam, Gortex, nonwovens (e.g., meltblown, spunbond, etc.), coated woven textiles, cotton, wool, felt, natural and synthetic leather, natural and synthetic rubbers, and the like, and combinations thereof. Preferably, such sheath member 110 will have a flexibility that is at least equivalent to, or greater than, that of the therapeutic member 300, though it need not be. The optional sheath member 110 can comprise any functional thickness, but will typically be relatively thin, such as between about 0.5 mm and about 10 mm. However, it should be understood that an optional sheath member 110 can have a thickness of less than 0.5 mm or greater than 10 mm without departing from the scope of the invention.

The invention also includes a method for making a therapeutic member 300. One non-limiting exemplary method can include:
A. Providing a suitable mitigation member 310;
B. Optionally processing the mitigation member 310 to create a plurality of apertures 320 and channels 322 generally in the z-direction 3 therewithin;
C. Providing a suitable mold (not shown) having inside dimensions in the x-y plane that are approximately equal to the mitigation member 310;
D. Optionally aligning and placing a barrier layer 340 in a laid-flat configuration into the mold;
E. Aligning and placing of the mitigation member 310 into the mold such that the bottom side 312 is disposed upon the optional barrier layer 340;
F. Preparing a suitable liquid polymeric reaction media;
G. Disposing a quantity of the liquid reaction media upon the top side 311 of the mitigation member 310 (preferably such that the reaction media is relatively evenly disposed thereon);
H. Optionally placing a comfort layer 350 in a laid-flat configuration upon the top side 311 of the mitigation member 310 (in contact with the reaction media);
I. Allowing the reaction media to fully cure into a thermal-conductive polymer 330; and
J. Removing the combination (i.e., mitigation member 310, thermal-conductive polymer 330, optional barrier layer 340 and optional comfort layer 350) to form an inventive therapeutic member 300 of the present disclosure.
   In some aspects, the method can optionally include applying a sheath member 110 to the therapeutic member 300 to at least partially encase the therapeutic member 300. It should be understood that the method can be modified to eliminate the use of a mold. In such embodiments, the bottom side 312 of the mitigation member 310 (or an optional barrier layer 340) can merely be placed onto a suitably flat surface.

The invention of the present disclosure also includes a second method of making an inventive decubitus prevention device 100. In this embodiment, the method can include:
A. Providing a thermal support member 120 as described above;
B. Providing a massage member 150 as described above;
C. Providing a therapeutic member 300 as described above;
D. Disposing the bottom side 122 of the thermal support member 120 upon the top side 151 of the massage member 150;
E. Disposing the bottom side 302 of the therapeutic member 300 upon the top side 121 of the thermal support member 120 to form an inventive decubitus prevention device 100 of the present disclosure; and
F. Optionally at least partially encasing the decubitus prevention device 100 in a sheath member 110.

The present invention may be better understood with reference to the following examples.

### EXAMPLES

### Example 1

A massage member 150 was constructed and provided. The massage member 150 comprised a housing component 160 comprising a steel outer frame element 162 (which generally formed the perimeter of the housing component 160) which had a length of about 190.5 cm, a width of about 99 cm and a height of about 18 cm. The housing component 160 also comprised a plurality of steel cross member elements 164 which extended across the width of the frame element 162 and which were generally attached to the bottom portions of the frame element 162 to provide additional structural support to the housing component 160. Extending downward proximate each corner of the frame element 162 were optional steel elevation elements 166 (i.e., legs), each of which had a length of approximately 17 cm, thus raising the bottom side of the housing component 160 approximately 17 cm above the floor. Disposed horizontally and lengthwise (i.e., along the x-axis 1) within the housing component 160 were a plurality of first manipulation elements 170A in the form of camshafts, each having a plurality of cams extending therefrom. These first manipulation elements 170A were rotationally mounted into bearings located at the “head” end and “foot” end of the frame element 162 and were additionally secured via collars mounted to the cross member elements 164. In addition, disposed vertically and along the width of the housing component 160 (i.e., along the y-axis 2) proximate a location where the lower back/buttocks portion of a user would be positioned during use of the inventive decubitus prevention device 100 was a plurality of second manipulation elements 170B in the form of wheels having roller balls disposed around the top side circumference thereof. The second manipulation elements 170B were rotationally mounted into bearings located in the cross member element 164 disposed at that location. Both the first manipulation elements 170A and the second manipulation elements 170B could be engaged via electric motors, and could be controlled via a suitable wired and/or wireless controller. Accordingly, an exemplary inventive massage member 150 of the present disclosure was provided. The massage member 150 of this Example 1 was similar to the massage member 150 shown in FIGS. 3A-3B.

A generally rectangular mold having an open top side (not shown) was then provided. The mold had rounded corners and resembled a raised frame-like structure, and comprised an inside dimension having a length of approximately 190 cm, a width of approximately 98.5 cm and a height of approximately 30 cm. Disposed through the “foot” end side of the mold was a first circular aperture and a second circular aperture. Each aperture had a diameter of about 2.5 cm, and each was generally transversely aligned with each other at a height of approximately 20.5 cm from the bottom side of the mold to the center point of each aperture. In addition, the first aperture was located approximately 15 cm from one corner portion of the mold, and the second aperture was located approximately 25 cm from the same corner portion of the mold (i.e., the distance between the center points of the apertures was approximately 10 cm).

An optional bottom side barrier layer 136 was placed in a laid-flat configuration into the bottom portion of the mold. The barrier layer 136 comprised polypropylene and had a length of about 190 cm, a width of about 98.5 cm, and a thickness of about 1 mm.

A thermal element 140 was then provided. The thermal element 140 comprised 2.5 cm outer diameter PEX tubing and had a rounded zigzag overall shape terminating with two (2) inlet/outlet end portions, similar to that shown in FIG. 2A. An optional thermal element support component 144 in the form of a rectangular piece of standard mattress foam having a length of approximately 160 cm, a width of approximately 69 cm and a height of approximately 19 cm was then placed in a laid-flat configuration upon the top side of the barrier layer 136 within the interior of the mold and was generally centered therein. The thermal element 140 was then aligned with, and carefully laid upon, the top side of the support component 144 and the position of the combination (i.e., the thermal element 140 and the support component 144) was adjusted such that the end portions of the thermal element 140 were inserted into, and extended through, the respective first circular aperture and second circular aperture by about 1 cm from the exterior of the mold to form extended portions 142 thereof. It was observed that the PEX tubing extending through the circular apertures in the mold each had a snug fit within the apertures (to prevent leakage of liquid reaction media).

A sufficient quantity of a liquid polymeric reaction media was prepared. The reaction media comprised about 7.5 wt % methylene diphenyl diisocyanate based glycol prepolymer (ELASTOCAST TQZP23 available from BASF Corporation), about 27 wt % hydroxyl terminated polybutadiene resin (POLY BD R45 HTLO, available from Cray Valley), about 65 wt % epoxidized soybean oil plasticizer and about 0.5 wt % slow-acting organobismuth catalyst (COSCAT 83 available from Vertellus Holdings LLC). Immediately upon thoroughly mixing the constituents, the liquid reaction media was poured into the mold until the height of the reaction media reached approximately 25.5 cm as measured from the bottom of the mold. While the reaction media was still in a partially-cured state, an optional top side barrier layer 138 was aligned with, and placed in a laid-flat configuration upon, the top surface of the reaction media. The top side barrier layer 138 comprised polypropylene and had a length of about 190 cm, a width of about 98.5 cm and a thickness of about 0.5 mm. The reaction media was then allowed to fully cure into a cushioning polymer 132 to form a polymeric member 130, thus providing an exemplary inventive thermal support member 120 of the present disclosure.

The resulting thermal support member 120 was removed from the mold, and was then aligned with, and placed upon, the massage member 150, thus providing a non-limiting exemplary embodiment of an inventive decubitus prevention device 100 of the present disclosure. The motors of the manipulation elements 170 were then electrically connected, and the ends (i.e., extended portions 142) of the thermal element 140 were connected to a thermal device 200 in the form of a heat pump (which utilized water). The inventive decubitus prevention device 100 of this Example 1 was then utilized by a user in a prone position. It was observed that the inventive decubitus prevention device 100 of this Example 1 seemed to reduce the feel of pressure points upon the user's body, and it was further observed that the inventive decubitus prevention device 100 successfully provided a therapeutic massage effect, a heating effect, and a cooling effect, as desired.

### Example 2

An inventive message member 150 was prepared and provided as described in Example 1. Likewise, an inventive thermal support member 120 was prepared and provided as described in Example 1. In addition, a generally rectangular mitigation member 310 (with rounded corners similar to those of the thermal support member 120 of Example 1) was provided. The mitigation member 310 comprised 1.5 pound per cubic foot/17 ILD, open-cell polyether foam (4200-245848, available from American Converters) and had a length of approximately 190 cm, a width of approximately 98.5 cm and a height (i.e., thickness) of approximately 2.5 cm. The mitigation member 310 was provided with a plurality of perforations in the form of circular apertures 320 which formed uniform cylindrical channels 322, each having a diameter of approximately 3 mm. The channels 322 extended entirely through the mitigation member 310 (from the top side 311 through the bottom side 312 along the z-axis 3), and were evenly spaced apart by a distance of about 2.5 mm as measured between the center points in both the length direction (i.e., along the x-axis 1) and the width direction (i.e., along the y-axis 2).

An optional barrier layer 340 was placed was placed in a laid-flat configuration onto a suitable flat surface. The barrier layer 136 comprised a substantially clear and colorless polypropylene film substrate and had a length of about 200 cm, a width of about 100 cm, and a thickness of about 0.1 mm. The bottom side 312 of the mitigation member 310 was then substantially centered and aligned with the barrier layer 340, and placed onto the top side of the barrier layer 340.

A sufficient quantity of a liquid polymeric reaction media was then prepared. The reaction media comprised about 6.34 wt % methylene diphenyl diisocyanate based glycol prepolymer (ELASTOCAST TQZP23 available from BASF Corporation), about 10.83 wt % 2-functional polyether diol (ELASTOCAST C4057, available from BASF Corporation), about 32.33 wt % 3-functional polyether triol (ELASTOCAST C4018, available from BASF Corporation), about 50.05 wt % epoxidized soybean oil plasticizer, and a blended catalyst comprising about 0.30 wt % slow-acting organobismuth catalyst (COSCAT 83 available from Vertellus Holdings LLC) and about 0.15 wt % heat-activated tin thioglycolate catalyst (FOMREZ CATALYST UL-54 available from Momentive Performance Materials Inc.). Immediately upon thoroughly mixing the constituents, the liquid reaction media was poured, and relatively evenly spread, upon the entire top side 311 of the mitigation member 310. The quantity of reaction media applied was approximately 5 grams polymer per gram mitigation member. While the reaction media was still in a partially-cured state, an optional comfort layer 350 was aligned with, and placed in a laid-flat configuration upon, the top side 311 of the mitigation member 310 (and the reaction media thereupon) such that the bottom side of the comfort layer 350 was substantially in contact with both the reaction media and the top side 311 of the mitigation member 310. The comfort layer 350 comprised a soft, flexible, resilient thermoplastic foam (i.e., standard mattress foam) which had a length of about 190 cm, a width of about 98.5 cm and a thickness of about 1 cm. It was observed that the thickness of the reaction media disposed upon the mitigation member 310 decreased over time as the reaction media soaked into the mitigation member 310. The reaction media was then allowed to fully cure to form an inventive thermal-conductive polymer 330 of the present disclosure, thus providing an inventive therapeutic member 300 of the present disclosure. The excess portions of the bottom side barrier layer 340 were then trimmed to the dimensions of the mitigation member 310.

Upon inspecting the therapeutic member 300, it was observed that a layer 335 of the polymer 330 had formed upon the top side 301, having an approximate thickness of about 1 mm. Upon inspecting the bottom side 302 of the therapeutic member 300, it was observed that each of the channels 322 was substantially entirely filled with the thermal-conductive polymer 330, and that the diameter of the channels 322 had randomly expanded to between about 6 mm and 10 mm.

The bottom side 122 of the thermal support member 120 was aligned with, and placed upon, the top side 151 of the massage member 150, and then the bottom side 302 of the therapeutic member 300 was subsequently aligned with, and placed upon, the top side 121 of the thermal support member 120, thus providing another non-limiting exemplary embodiment of an inventive decubitus prevention device 100 of the present disclosure.

The motors of the manipulation elements 170 were then electrically connected, and the ends (i.e., extended portions 142) of the thermal element 140 were connected to a thermal device 200 in the form of a heat pump (which utilized water). The inventive decubitus prevention device 100 of this Example 2 was then utilized by a user in a prone position. It was observed that the inventive decubitus prevention device 100 of this Example 2 seemed to completely eliminate the feel of any pressure points upon the user's body, and it was further observed that the inventive decubitus prevention device 100 successfully provided a therapeutic massage effect, a heating effect, and a cooling effect, as desired. It was noted that the massage effect of the embodiment of this Example 2 seemed a bit less pronounced and more widely distributed as compared to the embodiment of Example 1. It was also noted that the heating effect and cooling effect of the embodiment of this Example 2 seemed generally equivalent to the embodiment of Example 1, but that the time required to reach the endpoint temperatures took several minutes longer. It was further noted that the embodiment of this Example 2 subjectively felt a bit more soothingly comfortable than the embodiment of Example 1.

It will be appreciated that details of the foregoing examples, given for purposes of illustration, are not to be construed as limiting the scope of the present invention. Although only a few exemplary embodiments of the present invention have been described in detail above, persons having ordinary skill in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of the present invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

1. A decubitus prevention device comprising a massage member and a thermal support member, each comprising a top side and a bottom side;
wherein the massage member comprises a housing component and at least one manipulation element, wherein the at least one manipulation element is disposed within the housing component;
wherein the thermal support member comprises a polymeric member and at least one thermal element, wherein the at least one thermal element is at least partially disposed within the polymeric member; and
wherein the thermal support member is disposed upon the top side of the massage member such that the bottom side of the thermal support member is in contact with the at least one manipulation element.

2. The decubitus prevention device of claim 1, wherein the housing component comprises a frame element and at least one cross member element.

3. The decubitus prevention device of claim 1, wherein the massage member further comprises at least one elevation element disposed upon the bottom side thereof.

4. The decubitus prevention device of claim 1, wherein the at least one manipulation element comprises a first type of massage member and a second type of massage member.

5. The decubitus prevention device of claim 1, wherein the polymeric member comprises a viscoelastomeric and cohesive cushioning polymer.

6. The decubitus prevention device of claim 5, wherein the cushioning polymer is formed from a reaction media comprising:
a. about 3 wt % to about 20 wt % isocyanate prepolymer or silicone prepolymer;
b. about 20 wt % to about 40 wt % polyols; and
c. about 40 wt % to about 80 wt % epoxidized triglyceride plasticizer.

7. The decubitus prevention device of claim 6, wherein the polyols comprise hydroxyl terminated polyols.

8. The decubitus prevention device of claim 6, wherein the polyols comprise a polybutadiene polyol.

9. The decubitus prevention device of claim 8, wherein the polyols further comprise a polyether diol.

10. The decubitus prevention device of claim 6, wherein the epoxidized triglyceride plasticizer is an epoxidized soybean oil plasticizer.

11. The decubitus prevention device of claim 6, wherein the reaction media further comprises about 0.001 wt % to about 5 w % catalyst.

12. The decubitus prevention device of claim 1, wherein the thermal support member has a 00 Shore Hardness of about 0 to about 30.

13. The decubitus prevention device of claim 1, wherein the thermal support member further comprises a thermal element support component disposed at least partially within the polymeric member such that the thermal element support component is in contact with the at least one thermal element.

14. The decubitus prevention device of claim 1, wherein the thermal support member further comprises a bottom side barrier layer disposed upon the bottom side thereof.

15. The decubitus prevention device of claim 1, wherein the thermal support member further comprises a top side barrier layer disposed upon the top side thereof.

16. The decubitus prevention device of claim 1, further comprising a thermal device component connected to the at least one thermal element.

17. The decubitus prevention device of claim 16, wherein the thermal device component comprises a wireless user interface.

18. The decubitus prevention device of claim 1, wherein the thermal support member is at least partially encased in a sheath member.

19. The decubitus prevention device of claim 1, wherein the massage member and the thermal support member are collectively at least partially encased in a single sheath member.

20. A decubitus prevention device comprising a massage member, a thermal support member and a therapeutic member, each comprising a top side and a bottom side;
wherein the massage member comprises a housing component and at least one manipulation element, wherein the at least one manipulation element is disposed within the housing component;
wherein the thermal support member comprises a polymeric member and at least one thermal element, wherein the at least one thermal element is at least partially disposed within the polymeric member;
wherein the therapeutic member comprises a mitigation member having a top side and a bottom side and a thermal-conductive polymer;
wherein the thermal support member is disposed upon the top side of the massage member such that the bottom side of the thermal support member is in contact with the at least one manipulation element; and
wherein the therapeutic member is disposed upon the top side of the thermal support member.

21. The decubitus prevention device of claim 20, wherein the housing component comprises a frame element and at least one cross member element.

22. The decubitus prevention device of claim 20, wherein the massage member further comprises at least one elevation element disposed upon the bottom side thereof.

23. The decubitus prevention device of claim 20, wherein the at least one manipulation element comprises a first type of massage member and a second type of massage member.

24. The decubitus prevention device of claim 20, wherein the polymeric member comprises a viscoelastomeric and cohesive cushioning polymer.

25. The decubitus prevention device of claim 20, wherein the cushioning polymer is formed from a reaction media comprising:
a. about 3 wt % to about 20 wt % isocyanate prepolymer or silicone prepolymer;
b. about 20 wt % to about 40 wt % polyols; and
c. about 40 wt % to about 80 wt % epoxidized triglyceride plasticizer.

26. The decubitus prevention device of claim 25, wherein the polyols comprise hydroxyl terminated polyols.

27. The decubitus prevention device of claim 25, wherein the polyols comprise a polybutadiene polyol.

28. The decubitus prevention device of claim 27, wherein the polyols further comprise a polyether diol.

29. The decubitus prevention device of claim 25, wherein the epoxidized triglyceride plasticizer is an epoxidized soybean oil plasticizer.

30. The decubitus prevention device of claim 25, wherein the reaction media further comprises about 0.001 wt % to about 5 w % catalyst.

31. The decubitus prevention device of claim 20, wherein the thermal support member has a 00 Shore Hardness of about 0 to about 30.

32. The decubitus prevention device of claim 20, wherein the thermal support member further comprises a thermal element support component disposed at least partially within the polymeric member such that the thermal element support component is in contact with the at least one thermal element.

33. The decubitus prevention device of claim 20, wherein the thermal support member further comprises a bottom side barrier layer disposed upon the bottom side thereof.

34. The decubitus prevention device of claim 20, wherein the thermal support member further comprises a top side barrier layer disposed upon the top side thereof.

35. The decubitus prevention device of claim 20, further comprising a thermal device component connected to the at least one thermal element.

36. The decubitus prevention device of claim 35, wherein the thermal device component comprises a wireless user interface.

37. The decubitus prevention device of claim 20, wherein the mitigation member comprises at least one aperture.

38. The decubitus prevention device of claim 37, wherein the at least one aperture is disposed upon the top side of the mitigation member and extends at least partially through the mitigation member to form at least one channel.

39. The decubitus prevention device of claim 38, wherein the at least one channel comprises the thermal-conductive polymer at least partially disposed therein.

40. The decubitus prevention device of claim 20, wherein the therapeutic member comprises the thermal-conductive polymer disposed upon the top side of the mitigation member.

41. The decubitus prevention device of claim 40, wherein the therapeutic member further comprises the thermal-conductive polymer disposed at least partially through the mitigation member.

42. The decubitus prevention device of claim 41, wherein the therapeutic member further comprises a layer of the thermal-conductive polymer disposed upon the top side of the mitigation member.

43. The decubitus prevention device of claim 20, wherein the thermal-conductive polymer is formed from a reaction media comprising:
a. about 2 wt % to about 20 wt % prepolymer;
b. about 1 wt % to about 65 wt % straight chain polyols;
c. about 3 wt % to about 50 wt % crosslinking polyols;
d. about 40 wt % to about 80 wt % epoxidized triglyceride plasticizer; and
e. 0 wt % to about 40 wt % viscosity reducing plasticizer.

44. The decubitus prevention device of claim 43, wherein the prepolymer is selected from the group consisting of isocyanate prepolymer and silicone prepolymer.

45. The decubitus prevention device of claim 43, wherein the straight chain polyols comprise polyether diols and the crosslinking polyols comprise polyether triols.

46. The decubitus prevention device of claim 43, wherein the epoxidized triglyceride plasticizer comprises epoxidized soybean oil plasticizer.

47. The decubitus prevention device of claim 43, wherein the viscosity reducing plasticizer comprises ester plasticizer.

48. The decubitus prevention device of claim 43, wherein the reaction media further comprises about 0.001 wt % to about 5 w % catalyst.

49. The decubitus prevention device of claim 20, wherein the therapeutic member further comprises a barrier layer disposed upon the bottom side thereof.

50. The decubitus prevention device of claim 20, wherein the therapeutic member further comprises a comfort layer disposed upon the top side thereof.

51. The decubitus prevention device of claim 20, wherein at least one of the thermal support member and the therapeutic member is at least partially encased in a sheath member.

52. The decubitus prevention device of claim 20, wherein the massage member, the thermal support member and the therapeutic member are collectively at least partially encased in a single sheath member.

53. A method of preparing a decubitus prevention device comprising:
a. providing a massage member having a top side and a bottom side comprising a housing component and at least one manipulation element, wherein the at least one manipulation element is disposed within the housing component;
b. providing a thermal support member having a top side and a bottom side comprising a polymeric member and at least one thermal element, wherein the at least one thermal element is at least partially disposed within the polymeric member; and
c. disposing the thermal support member upon the top side of the massage member such that the bottom side of the thermal support member is at least in partial contact with the at least one manipulation element to form the decubitus prevention device.

54. The method of claim 53, further comprising:
d. providing a therapeutic member having a top side and a bottom side comprising a mitigation member and a thermal-conductive polymer, wherein the thermal-conductive polymer is at least partially disposed within the mitigation member; and
e. disposing the therapeutic member upon the top side of the thermal support member.
